(19) United States
(12) Patent Application Publication
(10) Pub. No.: US 2024/0172946 A1
(43) Pub. Date: May 30, 2024

(54) SYSTEMS AND METHODS FOR MONITORING A VEHICLE CABIN

(71) Applicant: VAYYAR IMAGING LTD., YEHUD (IL)

(72) Inventors: HAREL GOLOMBEK, NETANYA (IL); ZIVI NADRI, MISHMAR DAVID (IL); NAFTALI CHAYAT, KFAR SABA (IL); MARIANA SARELY, NETANYA (IL); TSACHI ROSENHOUSE, KIRYAT ONO (IL)

(21) Appl. No.: 18/422,791

(22) Filed: Jan. 25, 2024

Related U.S. Application Data
(63) Continuation-in-part of application No. 17/033,801, filed on Sep. 27, 2020, now Pat. No. 11,925,446.
Application No. 17/033,801 is a continuation-in-part of application No. 16/983,004, filed on Aug. 3, 2020, now Pat. No. 11,033,194.
Application No. 16/983,004 is a continuation of application No. 16/282,650, filed on Feb. 22, 2019, now Pat. No. 10,729,339.
(60) Provisional application No. 62/689,260, filed on Jun. 25, 2018.
(60) Provisional application No. 62/633,676, filed on Feb. 22, 2018.
(60) Provisional application No. 63/056,629, filed on Jul. 26, 2020.
(60) Provisional application No. 62/906,182, filed on Sep. 26, 2019.
(60) Provisional application No. 63/441,246, filed on Jan. 26, 2023.

(51) Int. Cl. A61B 5/024; A61B 5/08; G01S 7/41; G01S 13/42

(52) U.S. Cl. CPC A61B 5/02444; A61B 5/02405; A61B 5/0816; G01S 7/41; G01S 13/42

(57) ABSTRACT

Vehicle cabin monitoring using a radar unit positioned within the cabin to obtain image data of the vehicle cabin, a beam deflector for deflecting electromagnetic waves into obstructed regions of the cabin and a processor to generate detect occupancy of seats within the vehicle cabin, categorize occupants, detect posture, determine seatbelt status and monitor life signs of the occupants.

### CROSS-REFERENCE TO RELATED APPLICATIONS

This application is a Continuation-In-Part of U.S. patent application Ser. No. 17/033,801 which was filed on Sep. 27, 2020 as a Continuation-In-Part of U.S. patent application Ser. No. 16/983,004 which was itself filed on Aug. 3, 2020, as a continuation of U.S. patent application Ser. No. 16/282,650 which was filed on Feb. 22, 2019, and which, in turn, claims priority and benefit from U.S. Provisional Patent Application No. 62/633,676, filed Feb. 22, 2018, and U.S. Provisional Patent Application No. 62/689,260, filed Jun. 25, 2018 and which further claims priority and benefit from U.S. Provisional Patent Application No. 62/906,782, filed Sep. 26, 2019 and U.S. Provisional Patent Application No. 63/056,629, filed Jul. 26, 2020.

This application further claims the benefit of priority from U.S. Provisional Patent Application No. 63/441,246, filed Jan. 26, 2023 the contents of which are incorporated by reference in their entirety.

### FIELD OF THE DISCLOSURE

The disclosure herein relates to systems and methods for radar based monitoring of the cabin of a vehicle. In particular, but not exclusively, the disclosure relates to detecting occupancy, posture and classification of occupants of vehicles and controlling the vehicle's system based on the monitored parameters such as mass or size or orientation of occupying objects.

The disclosure herein relates to systems and methods for enhancing radar reflections from objects. In particular, the disclosure relates to enhancing reflections to provide information regarding the objects from radio waves are reflected.

The disclosure herein relates to systems and methods for providing airbag suppression with radar systems using a skeleton model.

### BACKGROUND

For various reasons it is useful to know whether seats of a vehicle are occupied. For example, there is a limit to the number of passengers that a vehicle is allowed to carry. There are also size and weight limitations regarding different seats of the vehicle. Passengers under a certain age are not allowed to sit in front seats. Thus it is important to know how many occupants there are in a vehicle and where they are sitting.

In some jurisdictions, to encourage carpooling, private vehicles are allowed to use preferential roads such as bus-lanes if they are carrying at least a specific number of passengers in addition to the driver.

Moreover, there is particular need in the industry for vehicles to determine if and how to activate the airbag in case of an accident. Various criteria effecting whether or not an airbag is activated may include: age of the passenger, dimensions of the passenger, seat position and/or posture. For example, it is often recommended that the air bag should not be deployed for a one year old, or that the air bag should not be deployed if a passenger has legs on the dashboard or is leaning forward.

Car manufacturers utilize large number of sensors for monitoring the car performance and safety purpose. It is also useful to monitor the occupancy of the cabin and particularly whether seats of a vehicle are occupied and whether there are infants on board. For example, pressure sensors are deployed for detecting occupancy within the vehicle, position detectors to detect the configuration and position of the car seats, detector for baby left in the car, etc.

Wideband MIMO radar apparatus based on compact antenna arrays is currently used in various imaging applications to visualize near-field as well as far-field objects and characterize them based on their reflective properties.

Current state of the art techniques use MIMO radar signals to create 3D images. However, current MIMO imaging techniques lack the ability to achieve adequate resolvability when identifying targets in close proximity to each other or are found in a moving environment.

Complex target objects having different parts, each having their own mode of movement further complicate imaging with sufficient resolvability. Therefore, there is need to advance current MIMO radar imaging techniques to achieve a higher degree of resolvability.

Having a large number of sensors monitoring the inside of a vehicle causes expense and reliability issues. Indeed, some modern vehicles include a plethora of sensors for determining the occupancy of a vehicle. Further, these sensors do not identify and differentiate the passengers based on their age and body structure. These include sensors for identifying whether each seat is occupied, whether a baby is in a front seat so as to disable airbags and so on. It would be desirable to obtain the same information with a simpler system.

Thus, there is a need for a simpler system which replaces the vehicle sensors and operate safety systems according to the passenger's seating position. The invention described herein addresses the above-described needs.

### SUMMARY OF THE EMBODIMENTS

A first aspect of the embodiments is directed to vehicle cabin monitoring system including a radar unit comprising at least one transmitter antenna connected to an oscillator and configured to transmit electromagnetic waves into the vehicle cabin, and at least one receiver antenna configured to receive electromagnetic waves reflected by objects within the vehicle cabin and operable to generate raw data.

The system may further include at least one beam deflection device configured to direct the electromagnetic waves transmitted by the at least one transmitter antenna towards at least one shielded or obstructed region within the vehicle cabin and further configured to direct the electromagnetic waves reflected by objects within the shielded or obstructed region towards the at least on receiver antenna.

Where appropriate, the at least one beam deflection device may comprise an outbound radio beam generator comprising a radio wave deflector operable to redirect incoming radio waves into an outbound radio beam. Variously, the at least one beam deflection device may comprise a reflector, a refractor, a dielectric lens, a grating, a reflectarray, a time-varying reflectivity, a switched impedance, a controlled dielectric material, a movable element or the like as well as combinations thereof.

According to requirements, the at least one beam deflection device may be incorporated into a ceiling section of the vehicle cabin selected from a group consisting of a sunroof, a headliner, a roofing beam, a vehicle pillar, a roll bar and combinations thereof. Additionally or alternatively, at least one beam deflection device is incorporated into a windshield of the vehicle, a rearview mirror of the vehicle, a head restraint, at least one pillar of the vehicle or the like as well as combinations thereof.

Optionally, the at least one beam deflection device may further comprise a modulator operable to modulate incoming radio wave signals. By way of example, the modulator may comprise a temporal modulator operable to adjust reflectivity over time. Additionally or alternatively, the modulator comprises a temporal modulator operable to adjust gain over time according to a gain function or a temporal modulator operable to adjust beam-phase over time according to a phase adjustment function.

In various embodiments, the modulator may comprise the reflectivity modulation unit configured to adjust the reflectivity of the outbound radio beam generator so as to encode information therein.

According to other aspects a method is taught for monitoring a vehicle cabin. The method includes providing a radar unit comprising at least one transmitter antenna connected to an oscillator and configured to transmit electromagnetic waves into the vehicle cabin, and at least one receiver antenna configured to receive electromagnetic waves reflected by objects within the vehicle cabin and operable to generate raw data, providing at least one beam deflection device within the vehicle cabin; the at least one transmitter antenna transmitting electromagnetic waves into the vehicle cabin; the at least one beam deflection device directing the electromagnetic waves transmitted by the at least one transmitter antenna towards at least one shielded region within the vehicle cabin; objects within the at least one shielded or obstructed region within the vehicle cabin reflecting the electromagnetic waves; receiving electromagnetic waves reflected by objects within the vehicle cabin; and the at least one beam deflection device directing electromagnetic waves reflected by objects within the shielded or obstructed region towards the at least on receiver antenna.

The method may further include providing a processor unit configured to receive raw data from the radar unit and operable to generate image data based upon the received data; providing a memory unit configured and operable to store the image data; providing at least one output unit; and generating a set of complex values associated with voxels within the vehicle cabin including the shielded or obstructed region.

Where appropriate, the method may still further include converting the set of complex values into a 3D complex image; filtering the 3D images by removing contribution of at least one of sidelobes, multipath, thermal noise and clutter; and detecting occupancy of seats within the vehicle cabin.

Additionally or alternatively, the method for monitoring a vehicle cabin of further comprises categorizing at least one occupant of a seat within the vehicle cabin; detecting a posture of at least one occupant of a seat within the vehicle cabin; and determining a seatbelt status for at least one occupant of a seat within the vehicle cabin.

In particular embodiments, the method further includes the output unit cancelling air bag operation if unsafe.

### BRIEF DESCRIPTION OF THE FIGURES

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the various selected embodiments may be put into practice. In the accompanying drawings:

FIG. 1A illustrates a schematic illustration of a vehicle cabin showing where a radar sensor array may be situated to track position and movements of passengers;

FIG. 1B is a schematic flowchart illustrating an exemplary method for determining seat occupancy information of the vehicle according to an aspect of the invention;

FIG. 1C is schematic block diagram of the elements of an embodiment of the invention;

FIG. 2A is a schematic view of the ceiling of a vehicle having a sunroof to which a radar sensor array, typically a radar on chip is attached;

FIG. 2B is a schematic side view of a sunroof showing a radar chip embedded within the material of the sunroof;

FIG. 2C is a schematic side view of a sunroof consisting of upper and lower layers and having a radar chip encased between the upper and lower layers;

FIG. 2D is a schematic side view of a sunroof showing a radar chip attached to the lower surface of the sunroof;

FIG. 2E is a schematic side view of a sunroof showing a radar chip embedded in a cavity within the sunroof;

FIG. 3A schematically represents a radar chip embedded in a headrest;

FIGS. 3B schematically represents an embodiment of a reflecting beam deflecting radar sensing system;

FIGS. 3C schematically represents an embodiment of a refracting beam deflecting radar sensing system;

FIG. 3D illustrates the concept of beam-tilting reflector, such as a reflectarray;

FIGS. 3E and 3F represent an example of headliner-radome codesign and the achieved performance improvement;

FIG. 3G is a plot illustrating performance improvement due to joint co-design of the radar's radome and the headliner;

FIG. 4A is a block diagram indicating selected elements of a system for detecting a modulated reflection from an enhanced reflecting roadsign;

FIG. 4B is a flowchart representing a method for a radar unit receiving a modulated signal;

FIG. 5A illustrates a possible Van Atta array retroreflector according to another embodiment;

FIG. 5B represents a possible active variable wavelength specific retroreflector;

FIG. 6A is a flowchart illustrating the general operation of an embodiment of the system;

FIG. 6B and 6C illustrate exemplary in-position seating of passengers in the vehicle; and

FIG. 6D, 6E, 6F, 6G and 6H illustrate exemplary out-of-position seating of passengers in the vehicle.

FIG. 7 is a flowchart illustrating a method for determining seat occupancy information of the vehicle;

FIG. 8 is a schematic diagram of hardware employed in the MIMO detection system, in accordance with an embodiment of the invention;

FIG. 9A is an overall flowchart illustrating the processing steps employed, in accordance with an embodiment of the invention;

FIG. 9B is an overall flowchart illustrating general processing steps employed, in accordance with an embodiment of the invention;

FIG. 9C is a flowchart illustrating the processing steps employed in a first embodiment of the radar signal processing stage, in accordance with an embodiment of the invention;

FIG. 9D is a flowchart illustrating the processing steps employed in a second embodiment of the radar signal processing stage, in accordance with an embodiment of the invention;

FIG. 9E is a flowchart illustrating the processing steps employed in a third embodiment of the radar signal processing stage, in accordance with an embodiment of the invention;

FIG. 9F is a flowchart illustrating the processing steps employed in the target processing stage, in accordance with an embodiment of the invention;

FIG. 10A is a plot of radial displacement as a function of time, as measured for a human subject, in accordance with an embodiment of the invention;

FIG. 10B depicts two plots of spectral power density for two identified elements, in accordance with an embodiment of the invention;

FIG. 11A-E depict image products at various stages of processing of passengers sitting in a car interior environment, in accordance with an embodiment of the invention; and

FIG. 12 is a graph depicting operating stages of one operating cycle employed during active detection modes, in accordance with an embodiment of the invention.

FIG. 13 is a flow diagram of showing how a three dimensional complex radar image of the cabin of a vehicle may be used to extract data regarding which seats are occupied and for classifying the occupant of each occupied seat;

FIG. 14A is a flowchart illustrating a method for singular value decomposition filtering;

FIG. 14B is a flowchart illustrating a method for successive spatio temporal filtering;

FIG. 14C represent schematic illustrations of the filtering step;

FIG. 15A is a two dimensional mapping of the area around a central radar sensor, showing SVD components;

FIG. 15B shows the two dimensional mapping of the area around a central radar sensor after performing a DBSCAN clustering;

FIG. 16 shows the two dimensional mapping of the area around a central radar sensor after performing spectral clustering;

FIG. 17 represents clusters of points as Gaussians in a three-dimensional space;

FIG. 18 shows the clusters that apparently represent different occupants with the position of the seats of the vehicle cabin superimposed thereover;

FIG. 19 shows seat arrangements shows the arrangement of the seats corresponding to that of FIG. 9;

FIG. 20 shows intermediate positions between seats 3 and 4 and between seats 5 and 6;

FIG. 21 is a transition model, showing valid state transitions between the back seats of the vehicle;

FIG. 22 is a flowchart showing how occupants may be classified;

FIG. 23A shows a positive cov in the xy plane;

FIG. 23B shows a negative cov in the xy plane; and

FIG. 24 is a side view of seat 5, showing the upper and lower boundaries and forward and rearward most boundaries of the cluster of signals interpreted as being the occupant.

FIG. 25A schematically represents a vehicle mounted radar unit configured to sense objects in the region surrounding a vehicle;

FIG. 25B is a block diagram indicating selected elements of a possible radar system detecting a signal reflected from an enhanced reflector;

FIG. 26A and 26B illustrate a possible corner cube retroreflector according to an embodiment of the enhanced reflector to be attached to an object thereby enhancing its radar reflection;

FIG. 26C illustrates a possible corner cube retroreflector incorporating a Van Atta array retroreflector according to another embodiment configured to be attached to an object thereby enhancing reflection from that object

FIGS. 27A-G illustrate various examples of enhanced reflectors attached to various subjects;

FIG. 28A schematically illustrates a vehicle mounted radar detecting an enhanced reflection from a road sign;

FIGS. 28B-F represents a selection of enhanced roadsigns;

FIGS. 29A-C represent examples of wavelength specific retroreflectors; and

FIGS. 29D-F illustrate the expected reflections from the wavelength specific retroreflectors of FIGS. 29A-C.

### DETAILED DESCRIPTION

Embodiments of the invention use a single sensor to track both occupancy and movements within the cabin of a vehicle. For monitoring passengers within a vehicle cabin by a single sensor, a possible position for the sensor may be central to the cabin such that as much as possible of the cabin is within the target range of the sensor. Variously this may be situated in a ceiling, within a seat, within a headrest, embedded in a window, embedded in a sunroof, embedded in a light unit, or the like as described herein.

A sensor may be hidden behind the fabric lining the cabin or within the upholstery of the seats for example. Additionally, or alternatively, a sensor may be positioned outside the fabric in a housing.

The sensor unit may detect presence within a vehicle cabin, covering both the front and back seats. Using a single sensor unit in this manner eliminates the need for separate sensor units in each row or in each seat, and replaces other sensors, saving labor and wiring costs during manufacturing.

Other aspects of the disclosure relate to systems and methods for enabling a single sensor unit to cover the whole of a vehicle cabin including obstructed regions. In particular, systems are described which include beam deflection devices, such as reflectors, refractors or diffractors configured to direct the electromagnetic waves from a sensor towards at least one shielded or obstructed region within the vehicle cabin.

Aspects of the present disclosure relate to systems and methods for determining seat occupancy information in a vehicle using a radar sensor. The seat occupancy may provide information about passenger's age class on each seat and in-position or out-of-position. The information may help to operate safety devices and track any passenger left in the vehicle. Particular aspects relate to systems and methods for providing airbag suppression where necessary.

In various embodiments, the sensor and supporting data analysis, for example using a skeleton model, determines the size of each occupant, (volume and dimensions), location, posture, tracks their movements within the cabin of the vehicle and monitors vital signs. Thus, a single sensor arrangement provides multifunctional monitoring of the vehicle cabin and replaces a plethora or sensors hitherto required.

Still further aspects of the present disclosure relate to systems and methods for enhancing radar reflections from objects. Retroreflectors may be used to enhance radar reflections from objects such that the reflected signal has a higher intensity and carries more information back to the radar detector.

Retroreflector systems may be further used for providing information to radar enabled vehicles regarding the identity and location of roadside objects. Accordingly, various passive and active retroreflectors may be used to encode characteristic information into the reflected signal which may be received by the radar detector and decoded to obtain information communicated by the reflector to the radar.

As required, detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

In various embodiments of the disclosure, one or more tasks as described herein may be performed by a data processor, such as a computing platform or distributed computing system for executing a plurality of instructions. Optionally, the data processor includes or accesses a volatile memory for storing instructions, data or the like. Additionally, or alternatively, the data processor may access a non-volatile storage, for example, a magnetic hard-disk, flash-drive, removable media or the like, for storing instructions and/or data.

It is particularly noted that the systems and methods of the disclosure herein may not be limited in its application to the details of construction and the arrangement of the components or methods set forth in the description or illustrated in the drawings and examples. The systems and methods of the disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials are described herein for illustrative purposes only. The materials, methods, and examples are not intended to be necessarily limiting.

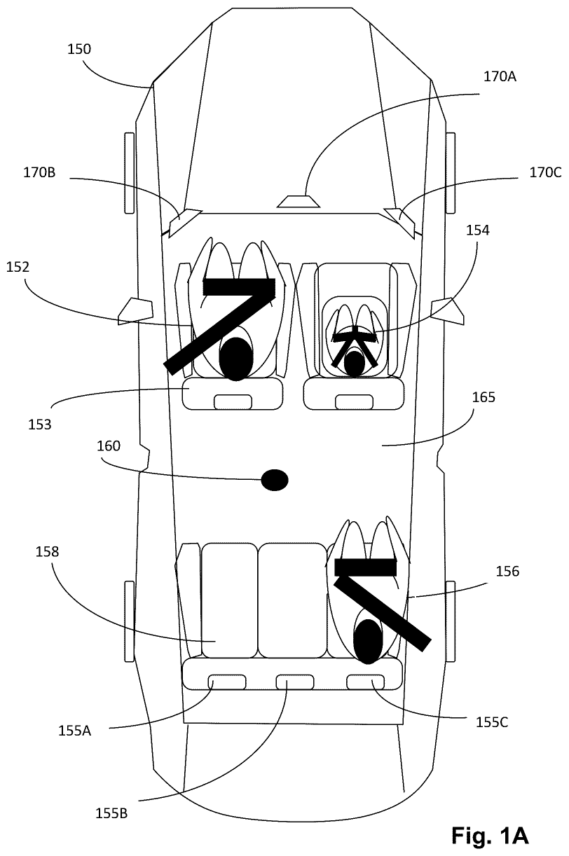

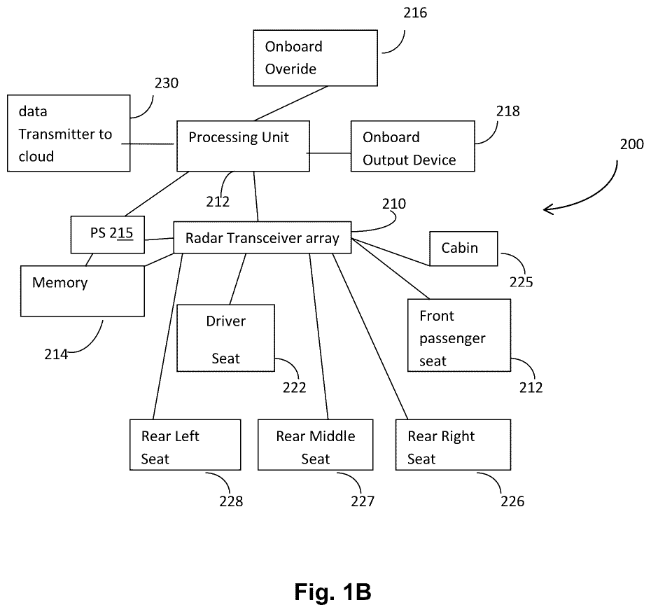

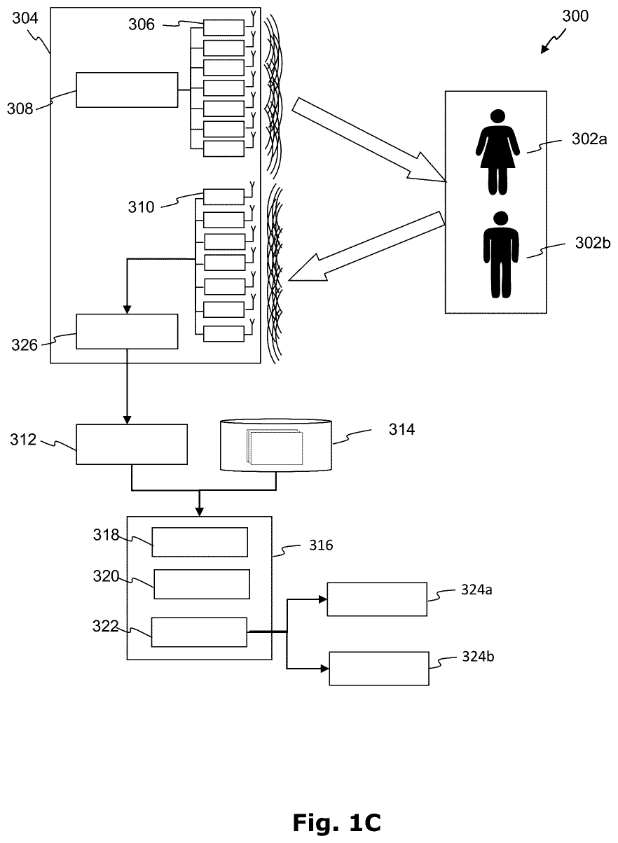

With reference to FIG. 1A, a schematic illustration of a vehicle 150 is shown. Within the vehicle, there is a driver 152, a child 154 sitting on the front passenger seat, a passenger 156 travelling on the back seat behind the front passenger seat and an empty seat 158 behind the driver.

Also shown, is a radar sensor array 160 internal to the cabin 165 of the vehicle and positioned centrally, such as on the ceiling of the vehicle 160, or alternatively within a seat, headrest or the like. The radar sensor array 160 monitors changes within the cabin 165 of the vehicle 150. As shown, the radar sensor 160 is centrally positioned. This is a preferred position as it may provide a field of view including the cabin, and good coverage of all the seats. Alternatively, the sensor may be positioned somewhat off-center and will still provide the information required for it to replace current sensors that are dedicated to particular individuals. Thus, the sensor array 160 may be installed at the top of the windshield or back window, for example. Where appropriate a double sided sensor may include forward facing and rear facing transceiver arrays thereby providing 360 degree coverage throughout the vehicle.

It is further noted that where regions of the vehicle cabin may be shielded or obstructed from view of the sensor array 160, additional beam deflecting elements 170A, 170B, 170C may be incorporated into the furniture of the vehicle cabin so as to direct electromagnetic waves into the regions which they do not reach directly. By way of example, beam deflecting elements may be incorporated into a front central position 170A such as within a rearview mirror, in other examples beam deflecting elements may be incorporated into side pillars 170B, 170C, still other examples may include beam deflecting elements such as reflectors, refractors or diffractors incorporated into headrests, seatbacks, headliners, flooring, sunroofs or the like.

The internal radar sensor array 160 may be an integrated system such as chip 160 described hereinabove. However it will be appreciated that in various embodiments, one or more tasks as described herein may be performed by an external data processor, such as a computing platform or distributed computing system of a vehicle, for executing a plurality of instructions. Optionally, the data processor includes or accesses a volatile memory for storing instructions, data or the like. Additionally or alternatively, the data processor may access a non-volatile storage, for example, a magnetic hard disk, flash-drive, removable media or the like, for storing instructions and/or data.

A preferred embodiment uses a radar sensor array that is integrated together with a digital signal processor (DSP) and a memory into a chip. For example, one embodiment uses a 4D imaging MIMO radar chip having global frequency bands (60 Ghz or 79 GHz), thousands of virtual channels, wide field of view on both axis and high resolution—angular and distance. The radar is provided on a chip (ROC) and preferred embodiments cover a dual-band range, supporting both 60 GHz and 79 GHz bands.

Another embodiment uses a sensor array that creates high-resolution images in real time based on advanced RF technology with radar bands from 3 GHz-81 GHz having 72 transmitters and 72 receivers integrated with a high-performance DSP with large internal memory that is capable of executing complex imaging algorithms without needing an external CPU.

Yet another embodiment has only 60-81 GHz, and 24 transmitter and 24 receivers.

As more sophisticated data analysis tools are developed, it is anticipated that the number of transceivers may be lowered, reducing unit cost, without reducing functionality.

Due to the integration of the number of transceivers and by sending, receiving and analyzing a multitude of signals with advanced DSP, high-resolution 4D images are obtained that track contours with high accuracy.

Many applications are directed to alerting the driver regarding occupants not using seatbelts, for locking doors and for disabling airbags deployed opposite seats with infants. The driver may also be provided with alerts of breathing or heart rate variations that of a passenger, or if the driver gets into difficulties, can be alerted to pull over, or in extreme cases, automatic control may take over.

In some embodiments, the radar system or an onboard computing system of the vehicle to which the radar system is in data communication with and is configured to transmit information over a data communication network, such as a cellular network to a remote database supported by a cloud such as the Internet of Things. This enables fleet operators and vehicle hire companies to monitor usage, or the police and emergency services to monitor occupancy and passenger states remotely.

Thus with reference to FIG. 1B, a generalized embodiment of the invention 200 consists of a radar transceiver array 210 that is powered by a power supply 215, typically from the electronic system of the vehicle. The radar transceiver array 210 sends and receives radar signals in all directions into the cabin 225 of the vehicle, and particularly towards the driver seat 222, the front passenger seat 224, the rear right seat 226, the rear left seat 228 and to the rear middle seat(s) 227, in vehicles designed to carry three passengers in the back seat.

The radar transceiver array 210 detects elements in all directions within the cabin 165 of the vehicle 20, and each detected element has a spatial component and a temporal component and by pulsed radar signaling, changes in position over time are detected.

The radar transceiver array 210 is coupled to a memory 214 for storing previous readings and by the spatial components of the detected signals, and changes over time, i.e. their temporal components, and/or possibly also includes a library of standard responses indicative of drivers and passengers of various sizes sitting in the various seats. The processing unit 212 is thus able to determine the presence of passengers in each direction and to differentiate between adults, children and babies, pets and inanimate objects by determining the size of the occupant, the height, whether the occupant is breathing or displaying a heartbeat and so on. Because passengers are expected to be found in specific positions, i.e. seats, usefully, responses from the direction of the various seats that are indicative of adults, children and infants or babies may be stored and this can limit the amount of processing required to obtain useful results.

The functionality described is by way of example. The sensor array gathers a large amount of data representing the cabin of the vehicle and its contents, and monitors changes over time. The processor can be provided with additional algorithms and procedures to analyze the image batches in different ways and to add additional functionality over time. Thus it is likely that as additional variables require monitoring for legal, insurance or other purposes, the existing, installed sensor and processor may be further programmed to extract the relevant parameters from the data.

An advantage of using radar signals for this purpose is that they are not blocked by most fabrics and by many non-fabric materials either. It will, however, be noted that despite being able to classify passengers as adults or children and to monitor vital signs, unlike optical cameras, the resolution is insufficient to infringe privacy.

Reference is now made to FIG. 1C, which is a schematic block diagram of a system for radar based monitoring in a vehicle. The system 300 includes a radar unit 304, a pre-processor unit 312, a database 314, a processing unit 316 and output units 324a and 324b.

The radar unit 304 is installed in the vehicle, e.g. a car. For monitoring objects such as passengers within the vehicle's cabin, the radar unit 304 should have direct line of sight with passengers, and in many cases, the optimal location may be the ceiling area, preferably center-ceiling.

The radar unit 304 includes an array of transmitters 306 and an array of receivers 310. The array of transmitters 306 may include an oscillator 308 connected to at least one transmitter antenna or an array of transmitter antennas. Accordingly, the transmitters 306 may be configured to produce a beam of electromagnetic radiations, such as microwave radiation or the like, directed towards in all directions in the cabin 165 of the vehicle 150, including the driver seat 153 and passenger seats 155A-C shown in FIG. 1A. FIG. 1C shows the electromagnetic waves transmitted towards exemplary passengers 302a and 302b. The receiver 110 may include an array of receiver antennas configured and operable to receive electromagnetic waves reflected from the body of the passengers 302a and 302b.

The radar receiver array 310 is coupled to a memory 326 which stores the signals received by receiver 310. The memory 326 also stores the previous readings and the spatial components of the detected signals, and changes over time, i.e. their temporal components, and/or possibly also includes a library of standard responses indicative of drivers and passengers of various sizes sitting in the various seats. Additionally or alternatively, a neural network may be trained to identify and categorize passengers, mapping them to classifications such as age category and in-position/out-of position states.

The previous information stored in the memory 326 is transferred to the pre-processing unit 312. The pre-processing unit 312 is thus able to determine the presence of passengers in each direction and to differentiate between adults, children and babies, pets and inanimate objects by determining the size of the occupant, the height, whether the occupant is breathing or displaying a heartbeat and so on. Because passengers are expected to be found in specific positions, i.e. seats, usefully, responses from the direction of the various seats that are indicative of adults, children and infants or babies may be stored and this can limit the amount of processing required to obtain useful results. It is further noted that where multiple heartbeats or breathing rates are detected in a single location, this may be an indicator that an infant baby, say, is being held by an adult even where the image of the infant may by masked.

The processing unit 312 of the system 300, whether integrated with the radar transceiver array 310 or in data communication therewith, and possibly part of the onboard computer of the vehicle may interact with an onboard output device 318 such as a warning light or an audible signal such as an alarm beep or a verbal message. For example, noting that the passenger is not strapped in with the safety belt, or that a passenger in the front seat 324 is too small to be safely strapped in. Additionally, the processing unit 312 may interact with an onboard override 316, for example, to cancel airbags where they would be dangerous, such as where an infant is in a front passenger seat 324, or to adjust seatbelt tension.

Additionally, the processing unit 312 may be coupled to a data transmitter 330 for transmitting data regarding occupancy to the cloud. This data may be used by fleet operators to monitor the number of passengers in a vehicle, and by emergency systems, etc.

The movements, breathing and heart-rate of each passenger may also be monitored.

Various systems and methods may be used for monitoring vital signs, such as breathing rates and heart-rates of passengers. By way of example, a radar sensor may receive energy signals reflected from objects within a target region such as the vehicle cabin, identify oscillating patterns within a target region such as the vehicle cabin indicative of the vital signs, and process the oscillating signals to isolate breathing signals, heart rate signals and the like.

In certain embodiments, a processor unit collates a series of complex values for each voxel representing reflected radiation for the associated voxel in multiple frames, accordingly, for each voxel a center point in the complex plane and a phase value for each voxel in each frame may be determined. In this way a smooth waveform representing phase changes over time for each voxel may be generated and a subset of voxels indicative of the periodic bio-parameter may be selected such that the required vital sign indices such as heart rate, heart rate variability, breathing pattern and the like may be obtained.

Examples of such systems are described in the applicants co-pending U.S. patent application Ser. No. 17/800,643, which is incorporated by reference herein in its entirety.

It is further noted that a radar sensor in the headrest of the driver's seat may be well positioned to monitor the driver's vital signs by measuring the reflected radiation from the back of the neck of the driver. In this way, the driver's health and alertness may be monitored in an ongoing fashion.

The sensor chip 160 and processing may be operated only for short periods following an event, such as a door closing, the vehicle accelerating or stopping, etc. Where applicable, the system may operate in pulsed mode in spurts, conducting time dependent sensing over several milliseconds, then calculating and analyzing the data over perhaps tens of milliseconds, but then being idle for maybe three or four times the period that the processor is actively sensing or calculating. This may be useful to conserve power and to prevent overheating by facilitating heat dissipation.

Where a four dimensional (4D) imaging MIMO radar is provided on a chip 10 that uses a sensor array that creates high-resolution images in real time based on advanced RF technology with radar bands from 3 GHz-81 GHz having 72 transmitters and 72 receivers integrated with a high-performance DSP with large internal memory that is capable of executing complex imaging algorithms without needing an external CPU, it will be appreciated that the processing generates heat that has to be dissipated.

By operating in pulsed mode and having intermittent downtimes, such as when activating for some tens or hundreds of milliseconds to send and receive radar signals, and then process the data, and then putting into an idling mode for tens or hundreds of milliseconds, heat can be dissipated somewhat, but there may still be a need to dissipate heat.

In many vehicles, the optimal position for the sensor chip 160 is on the ceiling, but slightly further forward to better monitor the driver's arms and feet, without the driver's seat obstructing. However, in some models of vehicles there is a sunroof at this position. Other models have panoramic glass ceilings.

For economical reasons, OEMs prefer to have the same positioning of sensors for all vehicle variations to simplify manufacturing and reduce installation costs. This can result in a sensor being positioned in a less than ideal position and being unable to collect some data as a result, or to nevertheless achieve full coverage, manufacturers may opt to install two sensors which doubles component and installation costs and increases the complexity and thus the adversely reliability. Additionally or alternatively, beam deflection elements may be introduced to direct electromagnetic waves into obstructed or shielded regions within the vehicle cabin thereby enabling full coverage.

An embodiment is directed to a sensor embedded in a glass component such as the sun-roof or panoramic ceiling, or alternatively in the windshield or back window of the vehicle. This allows a single installation location, maximizes performance and minimizes the costs incurred in installation across the various vehicle models.

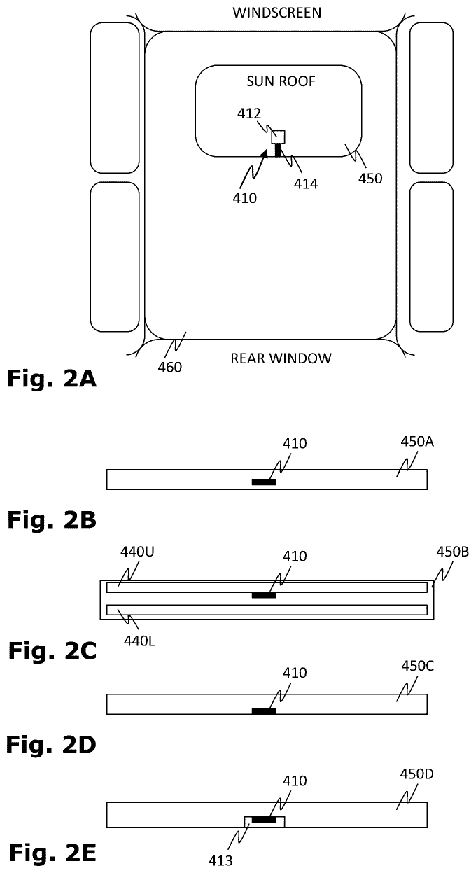

With reference to FIG. 2A, it has been surprisingly found that embedding or attaching the radar chip 410 (which may be the chip 160 of FIG. 1A and generally comprises radar transceiver array 412, memory 414, processing unit 412 and data inputs and a data transmitter to the cloud 230 as shown in FIG. 1B), to a sunroof 450 in the roof of a vehicle 460 such as an automobile or car aids in heat dissipation, as the large glass object conducts heat away from the chip.

Manufacturing sunroofs 450 with an embedded radar chip 410 makes installation of the radar chip 410 particularly easy. The wires 412 that connect the sensor to the vehicle to provide data and power, may also conduct heat away from the radar chip 410.

It will be appreciated that in some embodiments, the chip 410 is powered by a long term button magnet and does not require wiring at all. The chip 410 may be configured to transmit signals to the vehicle using very little power, since the transmission is short range. Furthermore, a solar panel may be provided on the chip 410 to recharge in daylight hours.

Sunroofs may be fabricated from glass or from a transparent polymer.

With reference to FIG. 2B, the chip 410 may be embedded in the sunroof 450A by casting the sunroof 450A around the chip.

Alternatively, with reference to FIG. 2C, the sunroof 450B may be fabricated from an upper layer 440U and a lower layer 440L of glass or other material, and the chip 410 may be positioned between two layers of glass for example the upper layer 440U and the lower layer 440L.

Alternatively again, as shown in FIG. 2D, the chip 410 may be adhered to the underside of the sunroof 450C.

As shown in FIG. 2E, a cavity 413 may be provided in a sunroof 450D and the chip 410 may be positioned in the cavity 410 and optionally held in place with a thermally conductive epoxy or thermoplastic that is transparent to the radar frequency and is preferably transparent.

The methods of attaching or embedding a radar transceiver sensor array or an integrated radar on chip described herein to a sunroof can be used to attach radar transceiver sensor arrays and radar chips to vehicles to monitor the near and distant areas outside the vehicle as well.

For example, a radar chip may be integrated into a headlamp, backlight or indicator light of a vehicle. These may be cast in glass or plastic, and are placed at the front and back of vehicles respectively.

A back facing radar may be useful when reversing or parking. The front facing radar may be useful when driving.

Modern car headlamp units and rear lamp units are typically large glass or plastic units that include a variety of lamps of different power and purpose, such as indicators, head lamp, dipped lamp, fog lamps, and so on. By incorporating the radar sensor array within this unit, it is easy to run wires to the radar unit and to avoid unsightly appendages.

Thus radar sensor arrays of the invention may be attached within glass headlamp units, mirrors, windshields and rear windows for monitoring the outside of the vehicle. Here again, a large glass or other heat conducting surface may act as a large heat sink, preventing the radar unit from overheating. Again, the radar sensor array may be integrated with a memory and digital signal processor into a chip that may be integrated into the headlight, rear light or indicator light unit, or into windows, such as the windshield, rear window or side windows of the vehicle, either by embedding into a cavity, or laminating between inner and outer layers, or simply adhered to an inner surface in a desired location and orientation.

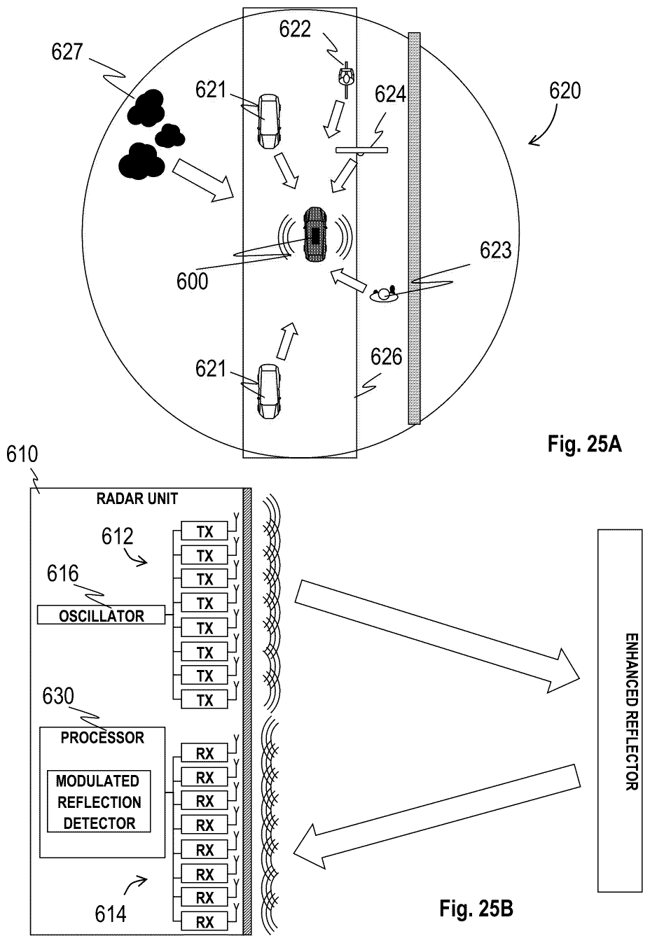

As noted hereinbelow in relation to FIG. 25A such external radar sensors may provide additional object sensing in the surroundings of the vehicle to which the radar sensors are mounted.

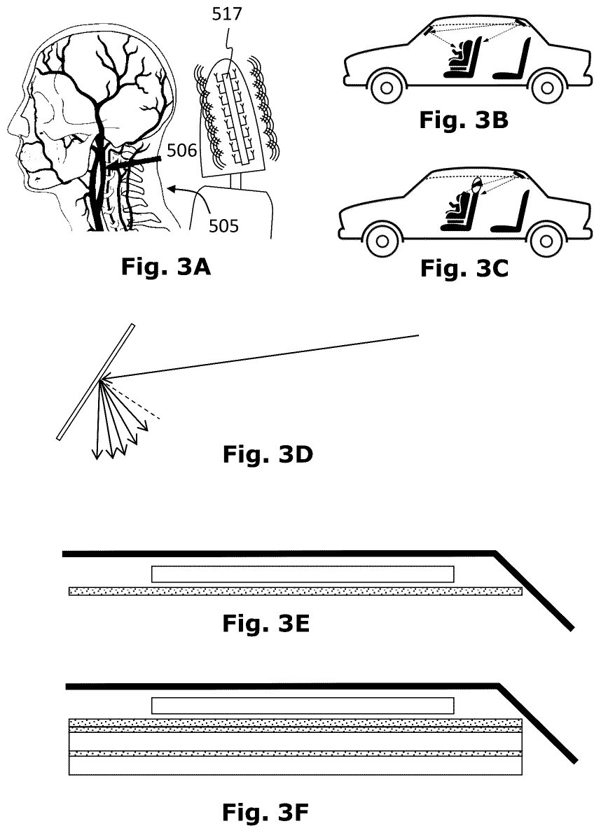

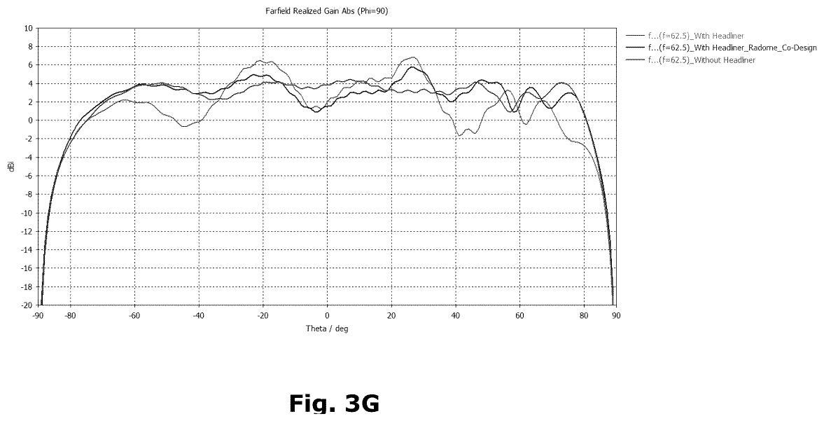

With reference now to FIG. 3A, it is further noted that another central position within the a vehicle may be the seats themselves. In particular the driver's seat or the passenger's seat in the front row of a two row cabin is roughly central to the cabin. Even in larger vehicle the front row headrests may provide a good central location for installing a radar sensor.

Accordingly, a radar sensing device incorporated into a headrest may be configured to transmit and receive in towards the front and the rear of the cabin. Various systems and methods may be used for providing a radar sensor with 360 degree coverage.

In particular it has been found that good heart rate signals have been observed when at least one sensor is directed towards the neck and upper back 505 of the subject. This may be due to the strong pulse passing through the carotid artery 506. Accordingly, as illustrated in FIG. 3A, it is noted that a sensor device 517 situated in a head rest of a car may be well positioned to monitor the life signs of the occupant of the car seat. Such a sensor may further monitor the wellbeing and alertness of the driver.

By way of example, a bidirectional radar sensor may include a Printed Circuit Board (PCB) mounted radar system having arrays of transmitting and receiving antennas mounted on an obverse surface of the PCB board to transmit and receive electromagnetic signals with communication devices which are located on the same side as the obverse surface. The system may further includes an array of receiving antennas mounted at the edge of the PCB to receive electromagnetic signals reflected towards the PCB from objects within the target region to the side of the PCB.

In other systems a reflector mounted on the PCB may be oriented such that the array of transmitting antennas transmit waves perpendicularly to the surface of the board which is incident upon the reflector surface and are directed radially away from the board, while the waves received from objects within the target region radially towards the reflector are directed towards the receiving antennas in a direction perpendicular to the PCB board. Where necessary phase shifters may be used to compensate for the different path lengths resulting from the reflected waves.

Control chips may be configured and operable to control all the active elements such as the antennas, reflectors and the phase shifters of the PCB including the transmitting and receiving antennas. Methods may be used to discriminate between targets on both sides of the PCB, when using the bi-directional antenna elements.

Examples of such systems are described in the applicants co-pending U.S. patent application Ser. No. 17/542,433, which is incorporated by reference herein in its entirity.

It is further noted that headset units incorporating radar sensors may be provided to be retrofitted to car seats. Such independent modules may include communication units for providing an interface with other modules such as the a computing unit, a mobile phone, an onboard infotainment system or the like. Where required, the headrest unit may further and independent power supplies such as electrochemical cells, solar panels, inductive power receivers and the like or may be configured to receive power from a vehicle power source.

Where regions of the vehicle cabin are shielded or obstructed from view of the radar sensor array, additional beam deflecting elements may be incorporated into the furniture of the vehicle cabin so as to direct electromagnetic waves into the regions which they do not reach directly.

Reference is now made to FIG. 3B, which illustrates one embodiment of a beam deflecting radar sensing system. The beam deflecting radar sensing system of the embodiment includes a radar sensor situated behind the headling of the cabin and a reflector situated at the upper corner of the vehicle cabin. Regions of the vehicle cabin such as the passenger seat may be obstructed from the radar sensor. In particular, the passenger seat back obstructs electromagnetic waves from reaching smaller objects upon the seat, such as a child seat or the like.

Accordingly, the reflector is provided to direct electromagnetic waves from the radar sensor towards the obstructed region and further to further to direct the electromagnetic waves reflected by objects within the obstructed region back towards the radar.

It will be appreciated that in other examples beam deflecting elements may be incorporated into other furniture such as the rearview mirror, side pillars, headrests, seatbacks, headliners, flooring, sunroofs a roofing beam, a vehicle pillar, a roll bar, a windshield, a window, a head restraint or the like as as well as combinations thereof.

FIG. 3D further illustrates the concept of beam tilting devices. Normally, flat surfaces reflect the waves according to specular reflection such that the angle of reflection is equal to the angle of incidence. However, by manipulating the local reflectivity phase across the surface, for example using a reflectarray of grating technique, the angle of reflection can be made different from the angle of incidence. This technology is useful when the reflector needs to be attached to a surface with a given direction and tilt. It is further noted that a selectively reflective grating may generate multiple angles of reflection for a given angle of incidence thereby providing further coverage of obstructed regions. Where appropriate, the multiple angles of reflection may be wavelength dependent such that multiple obstructed regions may be reached using a common beam deflector by selecting appropriate wavelengths of radio waves.

FIGS. 3E-G illustrate a preferred way to attach the radar sensor to the headliner of a vehicle cabin, behind which it is attached. Regularly, the radar sensor is equipped with a radome optimized for transmitting the radiated energy into the air. The radome typically takes the form of a dielectric cover of mostly uniform thickness, as illustrated in FIG. 3E. The thickness of the cover is typically of the order of half-wavelength in the dielectric material. However, this radome design causes performance degradation when the sensor is positioned behind the headliner of a vehicle cabin, which is itself composed of layers having dielectric properties, such as glass-epoxy composites, fleece, fabric, adhesive layers etc., as illustrated in FIG. 3F. The headliner is normally designed with attention to structural and aesthetic properties, but no attention to its radiowave transmission properties. Therefore, it is highly desired to modify the radome dimensions, and potentially the headliner dimensions, to improve the joint operation as the radome for the antenna array. The radome's thickness can be modified to improve its operation as a matching layer for the radiowaves to penetrate into the hedliner. The thickness of the headliner's layers may be locally adapted to improve wave transmission through the headliner. An adhesive layer or the like may be added. Local recess can be added, or local layer structure of the headliner may be modified. FIG. 3G shows an exemplary performance improvement due to joint co-design of the radar's radome and the headliner by varying the thicknesses of various layers. The severe ripple, over penetration angle, is nearly eliminated by the joint design.

In an embodiment, the penetration of radiowaves into the headliner can be improved by a stick-on layer attached to the headliner, serving as a matching layer. Similarly, a stick-on layer can be used to improve penetration properties of other dielectric surfaces in a vehicle. For example, a stick-on layer can be attached to a front windshield (or another window, such as a rearview window) of a vehicle, to serve as a better radome for a front-looking (or back-looking) radar mounted behind a windshield. The design of such stick-on layer (or a multilayer structure) would typically consider the typical thickness of the window, its tilt and the desired field of view of the radar. In an embodiment, the stick-on layer was found to improve the transmission in the out-of-car radar band (e.g. 77 GHz) while enhancing reflectivity for observing obstructed areas in the in-car radar frequency band (e.g. 60 GHz). The use of stick-on layers allows to retrofit existing vehicles with a radar so that the radar is better protected from the elements by virtue of its in-car placement.

Although a reflecting beam deflector is described above for the purposes of illustration, any beam deflection device which directs an incident radio beam away in a different direction may be used to increase cabin coverage. Other examples include refractors or diffractors such as dielectric lenses, gratings, reflectarrays and the like.

By way of further example, with reference to FIG. 3C a refracting beam deflector may be incorporated into the head restraint of the front seat so as to deflect the radio beam towards a child seat situated on the front seat in a region obstructed from direct line of sight from the radar.

It is further noted that beam deflection devices may enhance the radar beam in various ways, such as selectively reflecting particular wavelengths using wavelength specific retroreflectors or by providing time-varying reflectivity. Accordingly, beam deflection devices may include beam enhancement mechanisms such as switched impedance units, controlled dielectric materials or movable elements.

Such beam enhancement mechanisms may enable modulation of the incoming radio wave signals using modulators such as temporal modulators operable to adjust reflectivity over time, temporal modulators operable to adjust gain over time according to a gain function or to adjust beam-phase over time according to a phase adjustment function, or reflectivity modulation units configured to adjust the reflectivity of the outbound radio beam generator so as to encode information therein.

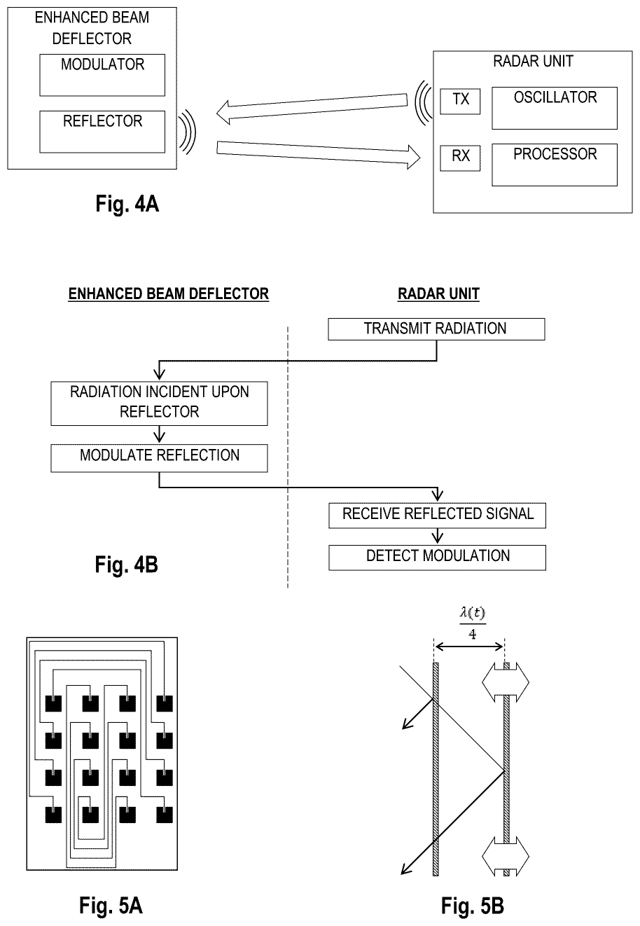

Referring now to the block diagram of FIG. 4A, the radar unit may include an oscillator, a transmitter, a receiver and a processor. The oscillator may produce a signal for example by sweeping through a range of transmission frequencies. The transmitter may transmit the signal to the surroundings. An enhanced reflector may include a modulator, such as a Van Atta array or the like, which increases the intensity of the reflection at a particular frequency characteristic of the reflector. The reflected signal may be received by the receiver or the radar unit.

With reference to FIG. 4B a method is illustrated for the interaction between the enhanced reflector and the radar unit receiving a modulated signal. The radar unit transmits radiation towards the reflector, the radiation is incident upon the enhanced reflector and the reflector modulates the signal of the reflected radiation. The enhanced reflected signal is received by the radar unit and the modulation is detected such that the information carried by the signal may be extracted.

Referring now to FIG. 5A, which illustrates an embodiment of a wavelength specific retroreflector. The wavelength specific retroreflector may include a Van Atta array retroreflector or the like. It is noted that it is a feature of a Van Atta array that only a particular bandwidth of incident radiation is reflected therefrom. It will be appreciated that wavelength specific retroreflectors may be selected such that the bandwidth of enhanced reflection may serve as coded information, for example it may be indicative of the identity of the object to which the retroreflector is attached.

Thus, for example, a radar may transmit radiation at multiple frequencies by sweeping through a frequency range. The radar detector may be configured and operable to detect the intensity of the reflected signal. Accordingly, when an enhanced reflected signal is detected, the frequency of the enhanced reflection may indicate the particular wavelength specific retroreflector producing the reflection. By carefully selecting the bandwidth of the wavelength specific retroreflector connected to a given body, coded information may be communicated to the radar perhaps pertaining to the identity of the body to which it is connected or the shielded region into which it deflects the radio beam.

Alternatively, an active variable wavelength specific retroreflector such as illustrated in FIG. 5B may be used to vary the reflector plate spacing as required. The variable wavelength specific retroreflector comprises two parallel planar radar reflectors spaced at a variable plate spacing λ(t)/4. Accordingly, incident radiation with a first wavelength of λ(t) is reflected strongly due to constructive interference and the enhanced wavelength λ(t) may be adjusted over time to encode various signals as required.

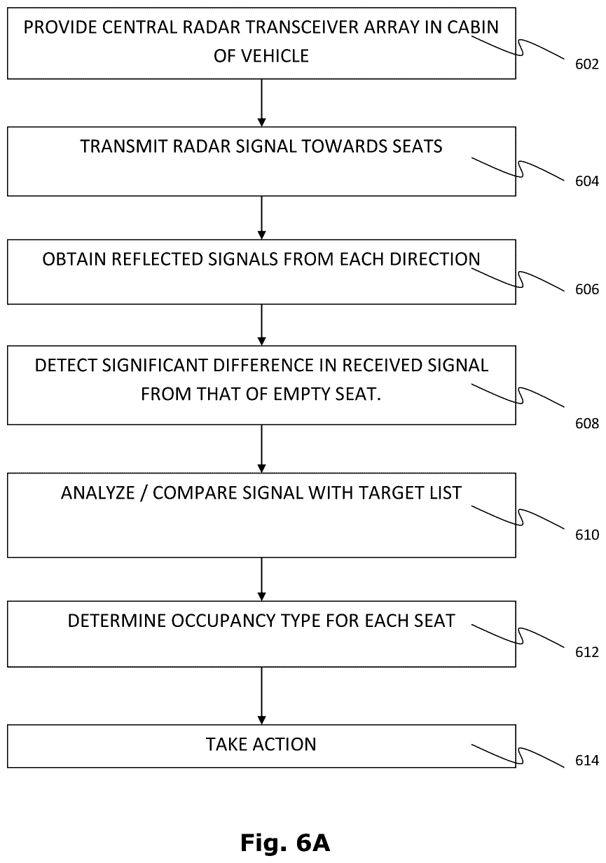

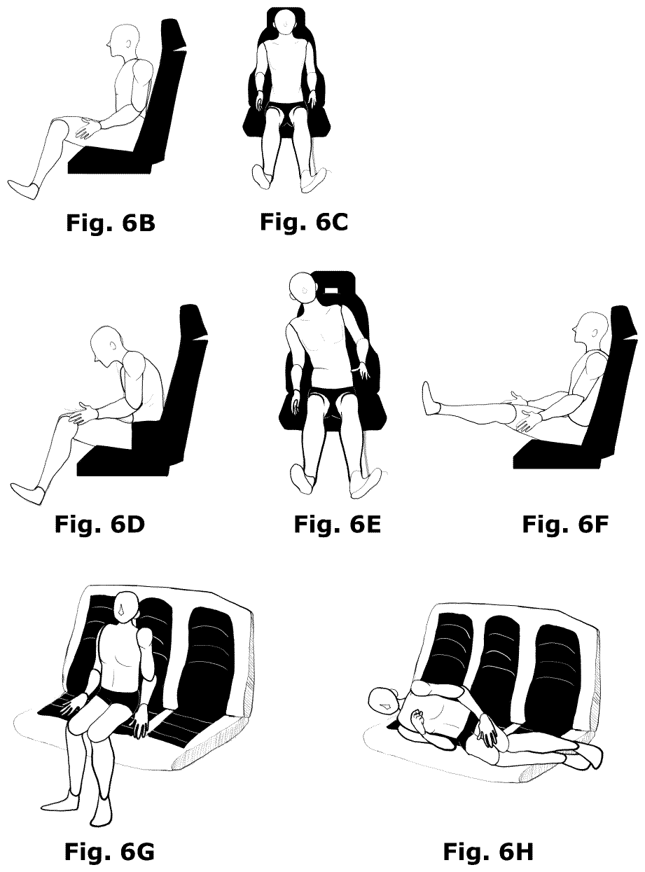

With reference to FIG. 6A, a generalized method for using the system of an embodiment is illustrated. Firstly, a central radar transceiver array is provided in the cabin of a vehicle—602. The central radar transceiver array transmits radar signals in all directions 604 and receives reflections from all direction 606, from the walls, seats and floor of the cabin, and from occupants, etc.

If a significant difference is detected between a received signal from the direction of a seat, and the signal expected from an empty seat 608, the signal may be analysed or compared with a signal for various targets 610, such as adults, children, pets, babies, and inanimate objects.

The system is also able to determine and categorize movements of each occupant, including heart beat and breathing, for example.

In this way the occupancy type for each seat may be determined 612 enabling the occupancy of each seat to be determined and categorized, and appropriate action 614 may be taken, such as alerting the driver with a warning light or an audible signal, over-riding, activating or disabling some component, such as an airbag, and/or transmitting a signal over a data network, such as a cellular network or the internet, for usage by fleet operators, emergency response personnel, family members of the driver and so on.

It is, however, particularly noted that the systems and methods of the disclosure herein may not be limited in its application to the details of construction and the arrangement of the components or methods set forth in the description or illustrated in the drawings and examples. The systems and methods in this disclosure may be capable of other embodiments, or of being practiced and carried out in various ways and technologies.

Alternative methods and materials similar or equivalent to those described herein may be used in the practice or testing of embodiments of the disclosure. Nevertheless, particular methods and materials described herein for illustrative purposes only. The materials, methods, and examples are not intended to be necessarily limiting. Accordingly, various embodiments may omit, substitute, or add various procedures or components as appropriate. For instance, the methods may be performed in an order different from described, and various steps may be added, omitted or combined. In addition, aspects and components described with respect to certain embodiments may be combined in various other embodiments.

The movements, breathing and heart-rate of each passenger may also be monitored as periodicity of movement may be identified as a breathing rate or a heart rate.

Various systems and methods may be used for monitoring vital signs, such as breathing rates and heart-rates of passengers. By way of example, a radar sensor may receive energy signals reflected from objects within a target region such as the vehicle cabin, identify oscillating patterns within a target region such as the vehicle cabin indicative of the vital signs, and process the oscillating signals to isolate breathing signals, heart rate signals and the like.

In certain embodiments, a processor unit collates a series of complex values for each voxel representing reflected radiation for the associated voxel in multiple frames, accordingly, for each voxel a center point in the complex plane and a phase value for each voxel in each frame may be determined. In this way a smooth waveform representing phase changes over time for each voxel may be generated and a subset of voxels indicative of a the periodic bio-parameter may be selected such that the required vital sign indices such as heart rate, heart rate variability, breathing pattern and the like may be obtained.

Examples of such systems are described in the applicants co-pending U.S. patent application Ser. No. 17/800,643, which is incorporated by reference herein in its entirity.

It is further noted that a radar sensor in the headrest of the drivers seat may be well positioned to monitor the driver's vital signs by measuring the reflected radiation from the back of the neck of the driver. In this way, the drivers health and alertness may be monitoried in an ongoing fashion.

The background signal indicative of an empty cabin is known. This is equivalent to the average signal, and may be subtracted from the detected signals to simplify analysis.

Signals may be clustered by synchronized movements, to identify individual passengers and to detect hand gestures and the like. An aspect of the invention is to provide a radar system that comprises a central sensor providing multidimensional time dependent tracking within the cabin of a vehicle for determining passengers within the cabin. Thus a RADAR sensor unit is located in the ceiling of a vehicle to monitors passengers in the front and back seats of the vehicle is shown. Typically the sensor operates in a pulsed mode.

By clustering response signals and mapping them with reference to the known position of seats in the vehicle, it is possible to limit the amount of processing required to detect and categorize passengers.

The sensor is coupled to a processing unit and is aware of the response from an empty seat which is a background response. By monitoring the difference in response from an empty seat and the detected signal and by comparison with a library of responses for babies, children and adults, the occupant of a seat may be classified as a baby, child, adult, pet or inanimate object.

Knowing the position of seats, enables detecting and classifying passengers more efficiently with less processing by applying a clustering algorithm and comparing with stored data. A box can quickly be drawn around each passenger, and used to determine how the passenger is sitting.

Movement of the chest indicating breathing or a heat beat being detected, may be used to differentiate between animate and inanimate objects which are sometimes put on and will reflect a different signal from that of an empty seat.

This information may be used to detect when a passenger is in difficulty or a baby is left behind in a vehicle, and after a crash to inform emergency personnel that there is life in a vehicle and in which seat. Where, after a crash, the breathing or heat beat of one or more passengers is detected intermittently or as labored, this information may be used by emergency personnel in deciding which passenger should be rescued and evacuated first.

The processing unit may use neural networks or fuzzy logic to determine nature of occupant.

The processing unit may be coupled to an indicator for indicating to a driver that a seat is occupied, for deploying airbags, for enabling remote tracking of passengers and for alerting emergency personnel in case of accident.

The central radar sensor can also determine changes to the cabin apart from the position of passengers. For example, if a door is opened.

The processing unit may by coupled to a database by cloud computing technology and configured to update database regarding seat occupancies.

Details of occupation of a seat may trigger an alarm if a seatbelt is not deployed.

If an occupant of a seat is indicated as too light or too short to be safely sitting in the seat, an appropriate notification may be made.

The information may be sensitive enough to enable smart seat belt pretensioners to adapt the seatbelts to the passenger.

The detection of a baby may operate a baby-on-board notification displayed or rear of vehicle to inform other drivers to keep their distance.

By knowing where passengers are seated and their size and shape enables airbag deployment in case of accident to be tailored to the position of the passengers. For example, an airbag will not be deployed in the front passenger seat if a baby is being carried. Additionally, airbags need not be deployed by empty seats. Knowing the position of the head of passengers and drivers enables optimization of airbag deployment, such as the selective deploying of airbags to better cushion the head on impact.

Moreover, the radar solution has the advantage that privacy is protected, as the identity of passengers is not detected.

The central radar sensor can also determine changes to the cabin apart from the position of passengers. For example, if a door is opened.

The processing unit may by coupled to a database by cloud computing technology and configured to update database regarding seat occupancies.

Some drivers are required by local law or by an owner of a fleet to be accompanied by a supervisor or a companion. For example, some military and police vehicle have rules prohibiting drivers of certain vehicles from traveling alone. Other vehicles are not allowed to carry passengers. For example, it may be useful to ensure that a new driver is accompanied by an adult or does not transport others.

In the past, a multitude of separate sensors has been provided, each configured replace to detect one thing, such as a pressure sensor in a seat, a head position sensor for deploying airbags, and so on. Advantageously, both from the perspective of unit cost and installation and wiring costs, the central sensor of the invention may replace a large number of such dedicated single task sensors.

However, it will be appreciated that a central sensor of the invention may be used together with other sensors, such as a biometric sensor to identify the driver of the vehicle. Where a thus identified driver should not travel alone or should only travel with an accompanying adult, the system could generate a warning to the driver, or inform a supervisor, or could be configured to prevent the vehicle from moving.

To decrease the number of vehicles and to incentivize car pooling, during times of peak congestion, some transport systems allow cars with 2, 3 or 4 occupants apart from the driver, to travel in lanes generally reserved for public transport. Embodiments of the invention track occupancy of vehicles and may be configured to provide occupancy levels via the cloud, to municipalities, traffic police and so on.

Indeed, even without special lanes, it is possible to use embodiments of the invention to monitor vehicle occupancy levels and this could be used to adjust tolls charged for usage of roads or bridges and the like, or to decrease annual road tax.

Knowing that a baby was being transported by a vehicle may be transmitted to the cloud so that in case of an accident, emergency workers know that a baby was being transported and will know to look for one. In this regard, a baby in a footwell or concealed by a blanket may be detected by its breathing or heart beats.

In addition to tracking location and height of passengers, preferred embodiments are configured to track posture, movements, particularly breathing of occupants and heart beats. This can be of value if an occupant gets into some sort of trouble. It can warn a driver to stop, for example.

After an accident the radar system may call emergency services and embodiments are operative to monitor vital signs and indicate these to emergency personnel over the cloud.

Additionally, in the case of an accident, the radar system may activate an audible alarm and announcement system.

By tracking occupancies, taxi services and bus owners can ensure that drivers do not conceal the number of passengers carried, illicitly pocketing profits.

It is also possible to track situations where two passengers share a seat or a passenger is standing. This can be useful for monitoring the number of passengers in driver vehicles or autonomous vehicles and may be used to prevent a vehicle from carrying more passengers than it is licensed to or legal for it to carry or for reporting such usage.

In the case of a carjacking or an abandoned vehicle, details of occupancy during a last journey by the vehicle may provide vital information for catching the perpetrators.

The system may be activated some minutes after a door is closed to detect children locked in vehicles.

By monitoring movement, breathing and cardiac activity of baby and alerting driver if something is wrong, the driver can concentrate on the road.

If the driver's breathing or cardiac activity show deviations from the norm, the driver can be advised to pull over and/or a signal can be transmitted to the cloud, enabling alerts to third parties such as a vehicle owner, a spouse, parent, child or next of kin, highway police, and so on.

Preferably, the system is able to detect gesture signals, enabling the driver and perhaps passengers to control various systems such as the radio, air-conditioning, and so on, via the processing unit.

Systems of the invention may be provided to vehicles driven by humans and to autonomous and semi-autonomous vehicles, and may be used by a driver to take over control from a semi-autonomous vehicle operating independently or for autonomous control to take over from a driver signaling for this to happen or indicating that he/she is undergoing some kind of health crisis, such as a heart attack or seizure.

Referring back now to FIG. 1C, the electromagnetic signals received by the receiver 310 are also sent to the pre-processing unit 312. The pre-processing unit 312 may be configured to extract Person Key Points (PKP) from the received signals using a trained deep neural network (DNN). The extracted person key points (PKP) may include:
Head—Center of top of the head
Left and Right shoulders
Left and Right external points of the lower abdomen or the pelvis.
Left and Right knees or points on the thighs

The extracted PKP are used to identify skeletal points of passengers at each seat of the vehicle. In a particular embodiment of the invention, the extracted key skeletal points may provide information in the following way:

The top of the head may give the highest point of the body which may be used for height calculation and for out-of-position detection.

The shoulders may allow determination of the width of the body.

The lower abdomen and pelvis may further delineate a square around the upper body allows getting determination of the actual sitting height. In addition, a base line may be created for the out-of-position check (abdomen/pelvis line vs shoulders line)

The knees and thighs may be used in addition to the abdomen and pelvis to assist determination, for example in cases where any of the other points are missing, obscured or not detected.

In addition, it might be possible to use the knee points to better differentiate between a child and an adult. In particular it has been surprisingly found that adult knees are generally detected above the plane of the pelvis since the legs reach the car floor. By contrast a child's knees are generally detected along or even below a line from the pelvis parallel to the floor.

The identified key skeletal points at each seat of the vehicle may be sent to a processing unit 316. The processing unit 316 includes a matching unit 318, a rules database 320 and a communicator 322. The matching unit 118 matches the key skeletal points received from the pre-processing unit 312 with standard passenger parameters received from the database 314. database 314 includes standard lists of PKP positions, distances and ranges in accordance with their age class. Table 1 illustrates exemplary class-age-height relation:

TABLE 1

| | | | | Typical | Typical |
|---|---|---|---|---|---|
| | | | | stature | sitting |
| | Meaning | Age | | height (cm) | height (cm) |
| NB | Newborn | 1 | month | 50 | — |
| IFT | Infant | 1 | years | 74 | 47 |
| SCD | Small Child | 3 | years | 94.5 | 54.5 |
| MCD | Medium Child | 6 | years | 115 | 63.5 |
| LCD | Large Child | 10 | years | 138 | 71.5 |
| ADT | Adult | >14 | years | >160 | >82 |

| | | | | |
|---|---|---|---|---|
| 5F | 5^{th} Percentile | — | 150 | 78 |
| | Female | | | |
| 50M | 50^{th} Percentile Male | — | 175 | 88 |
| 95M | 95^{th} Percentile Male | — | 188 | 92 |

As shown in Table 1, a child passenger of class MCD “Medium Child” has an age of about 6 years. Such a child has a typical stature height of about 115 cm and typical sitting height is about 63.5 cm. Similarly, an elder passenger of class ADT “Adult” has an age above 14 years. Its typical stature height is more than 160 cm and typical sitting height is more than 82 cm. It should be clearly noted that the class-age-height relation shown in Table 1 is exemplary in nature and should not limit the scope of the invention. The class-age-height relation varies according to the demography of each country and region.

It is further possible to add artificial Key Points by attaching reflective element to a seatbelt or to the seat itself. The reflective element can be a retroreflective element, such as a corner reflector, a Lunenberg lens, a catseye retroreflector or a PCB-based equivalent, to enhance the RCS of the reflective element. By way of example, a reflector on a seat can provide information whether it is obstructed by a human body. Reflection from the seat element may provide information about car vibration during ride. A reflector on a seatbelt can provide information whether a seatbelt is worn. Moreover, if the seatbelt is worn, the reflective element on a seatbelt becomes a PKP that can be used to better track respiration and heartbeat rerates signals by providing an augmented and more stable signal from a reflector moving in concert with the chest. The reflector may incorporate a modulating circuitry, e.g. using reflectarray technology, to impose a signature onto the reflected signal, to distinguish or to discriminate reflectors from each other and from other elements present in vehicle's cabin.

The matching unit 318 determines the occupancy information of each seat of the vehicle based on the comparison of key skeletal points with the standard class-age-height relationship as illustrated in Table 1. The matching unit 318 is basically a conclusion engine which comprises a heuristic code and/or trained machine learning solution. The conclusion outputted from the matching unit 318 may include: Occupancy per seat, Age class per occupant, or In-position or out-of-position detection per occupant.

The matching unit or conclusion engine 318 determines if a seat is occupied or vacant in the vehicle. As shown in FIG. 1A, the front seats are occupied by the driver and a passenger. The back seat is vacant, and another seat occupied by the passenger. In a particular embodiment, movement of the chest indicating breathing, or a heat beat being detected, may be used to differentiate between animate and inanimate objects which are sometimes put on and will reflect a different signal from that of an empty seat.

The matching unit or conclusion engine 318 may also determine the age class of each passenger in the vehicle. In FIG. 1A, age class of the driver may be determined to be “ADT”, an adult, the passenger may be a medium child of age class “MCD” and the passenger may be a fiftieth percentile man of age class “50M”.

The matching unit or conclusion engine 318 may also help to detect the in-position or out-of-position of each occupant. In-Position is defined as a normal sitting position of the passengers in the vehicle. FIG. 6B and 6C illustrate exemplary in-position seating of passengers in the vehicle. In the in-position or normal seating position, the passenger is seated with straight back and lying with the seat. The portion of legs above and below the knees are almost perpendicular to each other. FIGS. 6D-H illustrate various illustrative out-of-position seating of passengers in the vehicle.

The information of occupancy, age class and out-of-position thus determined may be transferred to the rules database 320 which determine actions for each seat based on the received information. The determined actions are then transmitted to the output units 324a and 324b through the communicator 322.

Few exemplary actions determined by the rules database 320 for a particular seat may include cancelling air bag operation if deemed unsafe, for example, a child is sitting in the front seat or someone has put their feet up on the dashboard, the strength of the seatbelt retraction may be adjusted to suit the size and posture of the occupant, alert may be sounded if a baby is left on board or an occupant is in an unsafe posture. The alert may be provided to an onboard output device 324a in form of a warning light or an audible signal such as an alarm beep or a verbal message.

As observed above, there is a need in the industry for vehicles to determine if and how to activate the airbag in case of an accident. Various criteria effecting whether or not an airbag is activated may include: age of the passenger, dimensions of the passenger, seat position and/or posture.

For example, air bag should not be deployed for a one year old. The air bag should not be deployed if a passenger has legs on dashboard or is leaning forward.

Current passenger detection solutions are generally based on weight sensors, however there is a large overlap between the weight ranges of heavy baby seats with babies and light females. This overlap limits the effectiveness of airbag cancellation functionality and in particular algorithms which take sensor readings as their inputs.

Furthermore weight sensors cannot estimate the posture and location of the passenger(s).

Embodiments of the current invention introduce an imaging radar that can create a point cloud, and extract a Skeleton Model from the point cloud which will be able to provide “joints coordinates” (“head, shoulder, knees, toes”) to the upper layer, which will enable modeling to accurately determine Dimension which are indicative of age as well as location and posture of the passengers and seats within the vehicle.

With that all current and future needs can be met.

The radar typically includes at least one array of radio frequency transmitter antennas and at least one array of radio frequency receiver antennas. The radio frequency transmitter antennas are connected to an oscillator (radio frequency signal source) and are configured and operable to transmit electromagnetic waves towards the target region. The radio frequency receiver antennas are configured to receive electromagnetic waves reflected back from objects within the target region.

Accordingly the transmitter may be configured to produce a beam of electromagnetic radiation, such as microwave radiation or the like, directed towards a monitored region such as an enclosed room or the like. The receiver may include at least one receiving antenna or array of receiver antennas configured and operable to receive electromagnetic waves reflected by objects within the monitored region.

The raw data generated by the receivers is typically a set of magnitude and phase measurements corresponding to the waves scattered back from the objects in front of the array. Spatial reconstruction processing is applied to the measurements to reconstruct the amplitude (scattering strength) at the three dimensional coordinates of interest within the target region. Thus each three dimensional section of the volume within the target region may represented by a voxel defined by four values corresponding to an x-coordinate, a y-coordinate, a z-coordinate, and an amplitude value.

Typically the receivers are connected to a pre-processing unit configured and operable to process the amplitude matrix of raw data generated by the receivers and produce a filtered point cloud suitable for model optimization.

Accordingly, where appropriate, a preprocessing unit may include an amplitude filter operable to select voxels having amplitude above a required threshold and a voxel selector operable to reduce the number of voxels in the filtered data, for example by sampling the data or clustering neighboring voxels. In this manner the filtered point cloud may be output to a processor. It is further note that the filtered point cloud may further be simplified by setting the amplitude value of each voxel to ONE when the amplitude is above the threshold and to ZERO when the amplitude is below the threshold.

The occupancy information from the matching unit 318 may be used to detect when a passenger is in difficulty or a baby is left behind in a vehicle, and after a crash to inform emergency personnel that there is life in a vehicle and in which seat. Where, after a crash, the breathing or heat beat of one or more passengers is detected intermittently or as labored, this information may be used by emergency personnel in deciding which passenger should be rescued and evacuated first.

Knowing that a baby was being transported by a vehicle may be transmitted to the cloud so that in case of an accident, emergency workers know that a baby was being transported and will know to look for one. In this regard, a baby in a footwell or concealed by a blanket may be detected by its breathing or heart beats.

In addition to tracking location and height of passengers, preferred embodiments are configured to track posture, movements, particularly breathing of occupants and heart beats. This can be of value if an occupant gets into some sort of trouble. It can warn a driver to stop, for example.

After an accident the radar system may trigger a communication system to contact emergency services and embodiments are operative to monitor vital signs and indicate these to emergency personnel over the cloud.

Additionally, in the case of an accident, the radar system may activate an alerting system to emit an audible alarm and announcement system.

By tracking occupancies, taxi services and bus owners can ensure that drivers do not conceal the number of passengers carried, illicitly pocketing profits.

It is also possible to track situations where two passengers share a seat or a passenger is standing. This can be useful for monitoring the number of passengers in driver vehicles or autonomous vehicles and may be used to prevent a vehicle from carrying more passengers than it is licensed to or legal for it to carry or for reporting such usage.

In the case of a carjacking or an abandoned vehicle, details of occupancy during a last journey by the vehicle may provide vital information for catching the perpetrators.

The system may be activated some minutes after a door is closed to detect children locked in vehicles.

Also, by monitoring movement, breathing and cardiac activity of an on-board baby and alerting a driver if something is wrong, the driver can concentrate on the road.

If the driver's breathing or cardiac activity show deviations from the norm, the driver can be advised to pull over and/or a signal can be transmitted to the cloud, enabling alerts to third parties such as a vehicle owner, a spouse, parent, child or next of kin, highway police, and so on.

The output units 324a and 324b may be an onboard output device such as a display or an audio output. Alternatively, the output units 324a and 324b may be remotely located in form of an external device, e.g., a client device, a server device, a routing/switching device or a cloud server. The communicator 322 may communicate the determined actions to the output units 324a and 324b through a network connection which may be a Wired LAN connection, a Wireless LAN connection, a Wi-Fi connection, a Bluetooth connection, a Zigbee connection, a Z-Wave connection or an Ethernet connection. For example, the communicator transmits the occupancy information and determined actions to the cloud server. This data may be used by fleet operators to monitor the number of passengers in a vehicle, and by emergency systems, etc.

One or more of the pre-processing unit 312, the database 314 and the processing unit 316 may be integrated within the system of the vehicle to process the information received from the radar unit 304. Alternatively, any of these units may be integrated with an external device, e.g., a client device, a server device, a routing/switching device or a cloud server. These units then communicate with the radar unit 104 through a network connection which may be a Wired LAN connection, a Wireless LAN connection, a Wi-Fi_33 connection, a Bluetooth connection, a Zigbee connection, a Z-Wave connection or an Ethernet connection.

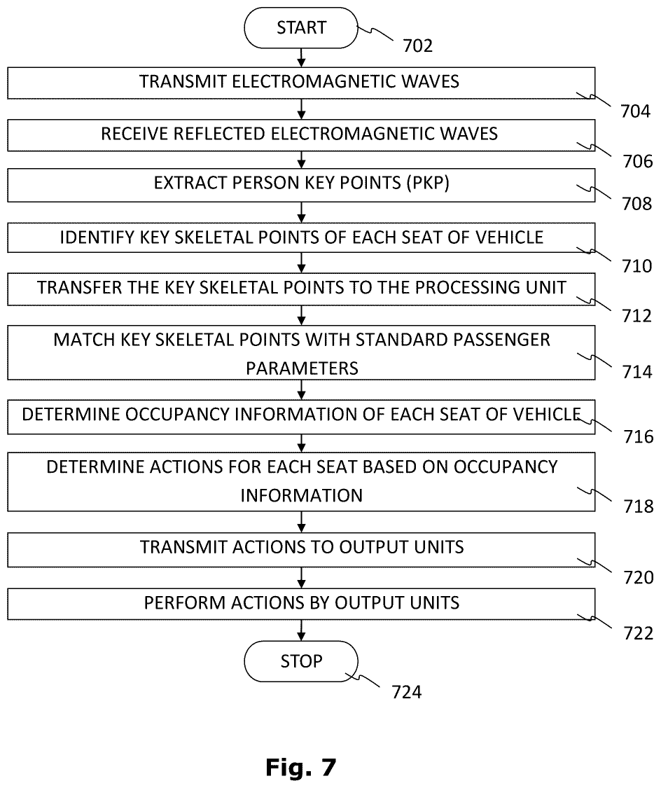

Referring now to FIG. 7 which illustrates an exemplary method for determining seat occupancy information of the vehicle 150. The process starts at step 702 and electromagnetic waves are transmitted by the transmitter array 306 towards the passenger seats 155A-C at step 704. The waves reflected from the passenger seats 155A-C at step 704 are received by the receiver array 310 at step 706. At step 708, person key points (PKP) are extracted by the pre-processing unit 312 from the received EM waves and key skeletal points are identified for each seat of the vehicle at step 710. The extracted person key points (PKP) may include the information of passenger's head, left and right shoulders, left and right external points of the lower abdomen or the pelvis and left and right knees or points on the thighs.

At step 712, the key skeletal points are transmitted to a matching unit 318 of the processing unit 316. At step 714, the matching unit 318 compares the key skeletal points received from the pre-processing unit 112 with standard passenger parameters received from the database 314. The database 314 includes standard lists of position and posture of the passengers in accordance with their age class. At step 716, the matching unit 118 determines the occupancy information of each seat of the vehicle based on the comparison of key skeletal points with the standard class-age-height relationship stored in the database 314. The conclusion outputted from the matching unit 318 may include occupancy per seat, age class per occupant and in-position or out-of-position detection per occupant.

The information of occupancy, age class and out-of-position is then transferred to the rules database 320 which determine actions for each seat based on the received information at step 718. At step 720, the determined actions are transmitted to the output units 324a and 324b through the communicator 322. The desired actions are performed by the output units 324a and 324b at step 722 and the process stops at step 724.

In the following detailed description, numerous details are set forth to provide a thorough understanding of the invention. It will be understood by those skilled in the art that the present invention may be practiced without these specific details. In other instances, well-known methods, procedures, and components have not been described in detail so as not to obscure the present invention.

Embodiments of the present invention provide for RF signal processing to detect and obtain measurements from one or more elements of at least one target object. In related embodiments, detection and measurement is furthermore done without having to isolate or identify the specific part or parts which contribute to the correlated movement.

The term “complex target” herein denotes a target object having one or more parts not necessarily distinguished by their respective reflective properties alone (herein denoted as their respective “reflectivities”); but rather, through their correlated movement or motion.

Identification of target objects is based on their elements having dissimilar modes of motion. Similarly, identification of target object from a background is achieved from the contrast of their respective modes of motion.

Some applications provide for further classification of human targets into categories such as “adult”, “infant”, for example.

Other applications provide identifying regions or parts of the body. The human body is modeled as a collection of rigid bodies (the bones) connected by joints. Rigid bodies have the property that all points on the surface move in a correlated way, since they are all combinations of the 6 degrees of freedom of the rigid body. In these embodiments of the invention the grouping of correlated motions into elements facilitate the identification of regions or parts of the body.

Other applications provide detecting and measuring of physical activities, including but not limited to: walking, running, jumping; coordinated movement of the limbs; carrying objects; head turning; hand gestures; changes in posture; and the like.

Further applications of the present invention provide detecting correlated movement of individuals in specialized environments featuring particular background characteristics and monitoring requirements, including, but not limited to: vehicle interiors and other moving platforms; hospitals and other medical and care facilities; and public venues, non-limiting examples of which include airports and other transportation stations; shopping centers, warehouses, and other commercial establishments; residential and office complexes; museums, theaters, and entertainment halls; parks, playgrounds, and stadiums; and institutions such as schools.

Additional applications of the present invention include: medical and health-related applications; security applications; crowd management applications; and vehicle safety and comfort applications.

According to various embodiments of the present invention, a complex target can include the human body. In these embodiments, the parts of the body include, but are not limited to: the head, the neck, the individual limbs, and the torso. In certain embodiments, physiological activities such as respiration and heartbeat are detectable and measurable, without having to isolate or identify the region of the body responsible for respiration and heartbeat (i.e., the torso).

The term “correlated movement” herein includes movement of one physical element of an object set relative to another, volumetric changes of the elements themselves, changes in orientation, position, shape, contour, or any combination thereof.

The term “measure” and its variants herein denote not only determining quantitative values (including multivariate values), but also analyzing the values, particularly variations in time, and making qualitative characterizations thereof.

The term “voxel element” refers to an entity that has been decomposed from a series of 3D images, each of the images associated with its respective frame.

It should be appreciated that terminology is context dependent. In the context of the physical arena the same terminology is employed when referring to the signal or logical representation of the same entity.

A non-limiting example of such a qualitative characterization involves the measurement of multivariate physiological data, such as the heartbeat and respiration of a subject. Not only can these physiological activities be detected and measured as raw data, but it is also possible to include, as a measurement, a qualitative assessment of the subject's current physical and mental condition based on the breathing rate, heart rate, and heart rate variability. Mental condition is meant to include awareness level, sleepiness, fatigue, anxiety, stress and anger, among other conditions.

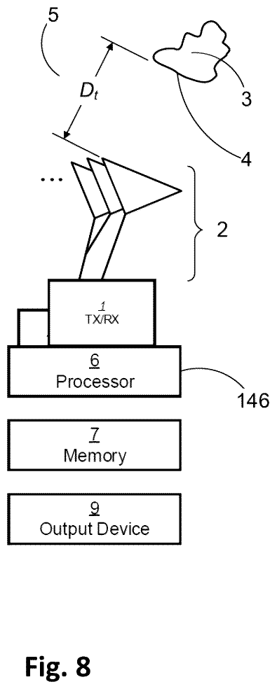

Turning now to the figures, FIG. 8 is a schematic block diagram of the MIMO imaging device including an antenna array 2 coupled to a radio frequency (RF) module 1 linked to a processor 6 in communication with memory 7 and output device 9, according to an embodiment. Output device 9 includes visual, audial devices, wireless devices, and printers.

As shown, the reflective elements of face 4 of target object set 3 provide differing reflectance as the radial distance Dt changes with time. Analysis of reflectance data in view of the reflectance data of previous time frames enables detection of correlated movement that advantageously provides discriminatory capability current unavailable in MIMO imaging systems. This is because traditional MIMO imaging systems repetitively construct images based on reflectance data of each time frame; independent of the reflectance data of the previous time frame. Accordingly, the use of correlated motion as differentiation tool constitutes an advance in the art of MIMO imaging.

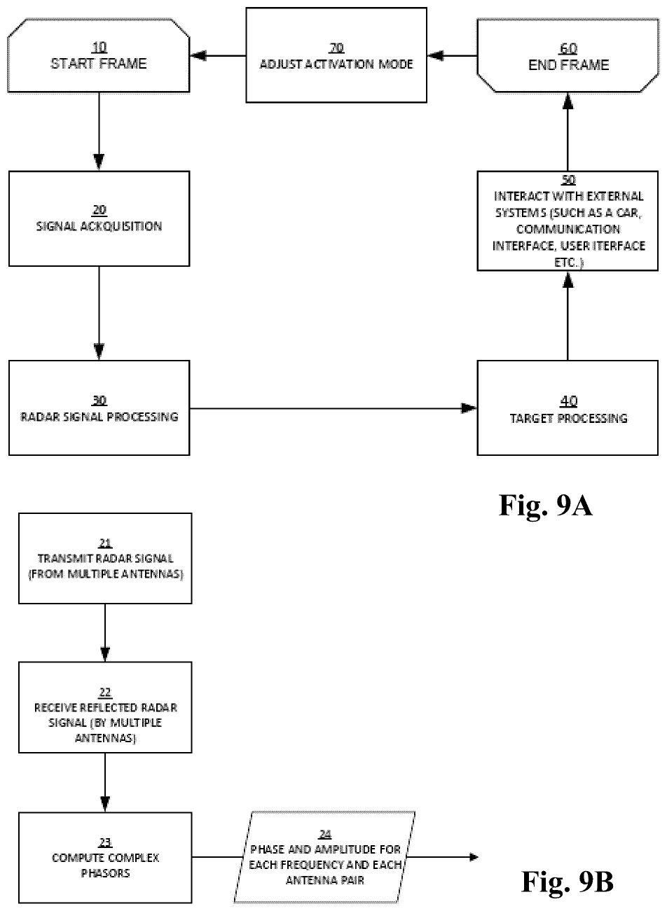

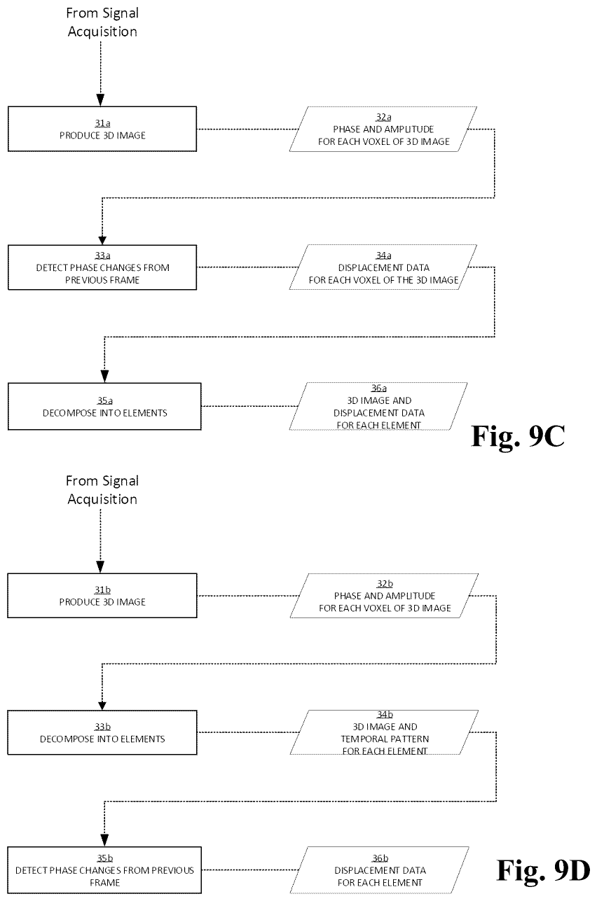

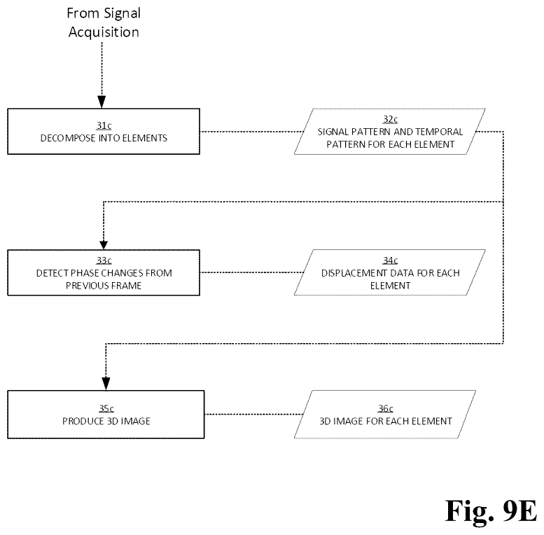

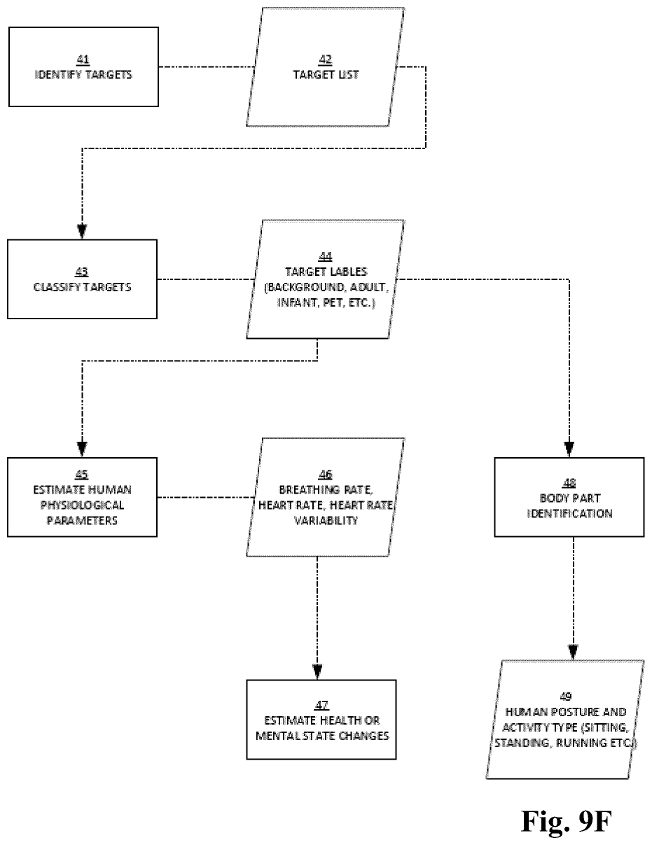

FIG. 9A is a high-level flowchart illustrating a general processing scheme according to an embodiment of the present invention. The scheme can be described as a closed loop, where each iteration of the loop consists of a sequence of steps.

The loop begins at step 10, where the acquisition and processing of a new time frame is started. Frames are started at regular intervals of Δt (meaning the frame rate equals

$$\frac{1}{Δ t} ) .$$

According to various embodiments of the invention, Δt is selected so that target movement ΔD during Δt is small compared to the wavelength of the radar signals

$$( i . e . ΔD ≪ \frac{c}{4 π f} )$$

maintain continuity from one frame to another. For waves having a central frequency f, the wavelength is c/f, where c is the speed of light. When detecting and measuring periodic correlated movement of the target, imaging by a series of frames is a sampling process, so that the frame rate should be set according to the Nyquist criterion to avoid aliasing. Frames are indexed by t=0, 1, 2 . . . corresponding to time, where successive indices represent respective multiples of a Δt.

In step 20 radar signals are transmitted, received and processed to produce complex phasors, representing the amplitude and the phase of the each received signal relative to each transmitted signals. Step 20 is further elaborated in FIG. 9B.

In step 30 several signal processing steps are performed, resulting in a set of components, each consisting of a spatial pattern and a trajectory (displacement vs. time). Step 30 is further elaborated in FIG. 4.

In step 40 components are used to identify targets, classify the targets and estimate target parameters of interest. Step 40 is further elaborated by FIG. 9C.

In step 50 the identified targets and their estimated parameters are used to interact with external systems, including, but not limited to, vehicle systems (e.g. to activate a horn, turn on air conditioning, unlock a door etc.), communication interfaces (e.g. to alert a user using his mobile device) or user interfaces (to inform users and allow them to take action).

In step 60 frame processing is ended. In step 70 the system's activation mode is adjusted according to timers, identified targets and their parameters, as well as user inputs. The system activation mode controls parameters including, but not limited to, the number of frames per second the system captures (which determines Δt) and the transmitting power. In some cases, the system is put in standby mode for a period of time. Activation mode adjustment is done in order to conserve system power. The loop closes when the next frame begins (according to the timing dictated by the activation mode), and the system returns to step 10.

FIG. 9B is a flowchart elaborating the RADAR SIGNAL ACQUISITION step from FIG. 7A (step 20). In Step 21, radar signals are transmitted from one or more antennas. If multiple antennas are used to transmit, the transmission can be done either sequentially (antenna-by-antenna) or simultaneously. In some embodiments of the invention antennas transmit simultaneously using a coding scheme such as BPSK, QPSK, or other coding schemes as is known in the art. Transmission may include a single frequency, or it may include multiple frequencies.

In step 22 the radar signals which have been reflected by targets in the physical environment surrounding the antennas are received by one or more antennas. Then in step 23 for each transmitted frequency and for each pair of transmitting and receiving antenna the received signals are processed to produce complex phasors, representing the phase and amplitude of the received signal relative to the transmitted signal (item 24).

FIG. 9C is a flowchart elaborating the RADAR SIGNAL PROCESSING step from FIG. 9A (step 30) in an embodiment of the invention. In step 31a a 3D image is produced from the set of complex phasors describing the received signal. The image space representation is conceptually summarized as a data block 32a containing an image matrix S=[S_{v,t}] with a voxel set V whose elements spatially conform to a system of coordinates. The particular system of coordinates for the voxel set can be chosen according to what is most convenient. Common choices include Cartesian coordinates (v_{x,y,z}) and polar coordinates (V_{r,θ,φ}), but any other coordinate system is equally usable. Each voxel is associated with a single value S_{v,t}=A_{v,t}e^{jϕ}^{^{v,t}} where A_{v,t} is the amplitude and ϕ_{v,t} is the phase associated with a reflector at voxel v. The phase ϕ_{v,t} is determined by the radial displacement of the reflector in voxel v from the center of that voxel (designated D_{v,t}). The phase is related to the displacement by the following formula:

$${ϕ}_{v , t} = \frac{4 π f}{c} {D}_{v , t}$$

where f refers to the central frequency. A single cycle extends over 2π radians, but an additional factor of 2 is needed because the reflection doubles the distance the waves travel.

In step 33a the value associated with each voxel at the current frame (S_{v,t}) is used together with the value associated with the same voxel at the previous frame (S_{v,t-1}), to obtain a robust estimate of the radial displacement between the two frames using the following formula:

$$= \frac{c}{4 π f} \frac{Im [ {S}_{v , t} {S}_{v , t - 1}^{*} ]}{\vert {S}_{v , t} \vert \vert {S}_{v , t - 1} \vert + λ {max}_{V} ( \vert {S}_{v , t} \vert \vert {S}_{v , t - 1} \vert ) + ϵ} \qquad ( 1 )$$

where λ and ϵ are real scalar parameters that are selected to minimize the effects of noise on the final value. Typical values for A and e are small, with reasonable values being about 0.1 for λ and about 1×10−8 for ϵ.

According to another embodiment of the invention a slightly modified version of the formula is used, in order to provide better linearity of the estimated displacement:

$$= \frac{c}{4 π f} Arg [ \frac{{S}_{v , t} {S}_{v , t - 1}^{*}}{\vert {S}_{v , t} \vert \vert {S}_{v , t - 1} \vert + λ {max}_{V} ( \vert {S}_{v , t} \vert \vert {S}_{v , t - 1} \vert ) + ϵ} ] \qquad ( 2 )$$

$$= \frac{c}{4 π f} Arg [ \frac{{S}_{v , t} {S}_{v , t - 1}^{*}}{\vert {S}_{v , t} \vert \vert {S}_{v , t - 1} \vert + λ {max}_{V} ( \vert {S}_{v , t} \vert \vert {S}_{v , t - 1} \vert ) + ϵ} ]$$

According to an embodiment of the invention, The estimated displacement data ( ) is recorded (item 34a) using a sliding window (which can be implemented, among other options by using a circular buffer), and in step 35a the radial trajectory component is decomposed into independent elements using Blind Signal Separation (BSS, also known as “Blind Source Separation”). In a related embodiment, the elements of the radial trajectory are separated by using Independent Component Analysis (ICA), a special case of BSS. In another embodiment, the elements of the radial trajectory are separated by Principal Component Analysis (PCA). In another embodiment, the elements of the radial trajectory are separated by Singular Value Decomposition (SVD).

In another embodiment of the invention, an online decomposition algorithm is used, avoiding the usage of a sliding window, allowing the separation of elements to be performed incrementally, frame-by-frame.

[symbol] is a matrix whose rows represent voxels, and whose columns represent frames. The decomposition algorithm extracts a factorization of [symbol] in the form of factor triplets (“elements”)

Cₖ=(u_{v,k}, σₖ, w_{k,t}) (3)

where the matrix [w_{k,t}] represents the aggregated frame-dependent (i.e., time-dependent) incremental radial displacements. And the matrix [u_{v,k}] represents a spatial (voxel-dependent) pattern associated with the component.

The incremental radial displacements are summed to obtain an estimated radial displacement trajectory as a function of time:

$$= {\sum }_{{t}^{′} = {t}_{0}}^{t} ( {w}_{k , {t}^{′}} {σ}_{k} {max}_{V} {u}_{v , k} ) \qquad ( 4 )$$

where the value is normalized to the largest observed incremental movement for the target. The term “summed” herein relates not only to a discrete representation in Equation (4), but also to “integration”, according to a related embodiment which calculates the trajectory as an integral.

The spatial pattern [u_{v,k}] and the radial displacement trajectory [symbol] are recorded as item 36a.

FIG. 9D is a flowchart elaborating the RADAR SIGNAL PROCESSING step from FIG. 9A (step 30) in an embodiment of the invention (separate from the embodiment described by FIG. 9C). In step 31b a 3D image is produced (item 32b), in a manner similar to the description hereinabove. In step 33b, the 3D image is decomposed using algorithms similar to the ones described hereinabove, producing a set of elements, each described by a 3D image and a temporal pattern consisting of complex phasors (item 34b). In step 35b each temporal pattern is processed using a phase detecting procedure similar to the one described hereinabove to produce displacement data for each element (item 36b).

FIG. 9E is a flowchart elaborating the RADAR SIGNAL PROCESSING step from FIG. 9A (step 30) in an embodiment of the invention (separate from the embodiments described by FIG. 9C and FIG. 9D). In step 31c the complex radar signal is decomposed using algorithms similar to the ones described hereinabove, producing a set of elements, each described by a complex time-independent signal pattern and a temporal pattern consisting of complex phasors (item 32c). In step 33c, each temporal pattern is processed using a phase detecting procedure similar to the one described hereinabove to produce displacement data for each element (item 34c). In step 35c, each time-independent signal pattern is used to produce a 3D image for the corresponding element (item 36c), in a manner similar to the description hereinabove.

FIG. 9F is a flowchart elaborating the TARGET PROCESSING step from FIG. 9A (step 40) in an embodiment of the invention. In step 41, elements are grouped into targets, representing detected physical objects, by examining the spatial pattern of each element, producing a target list (item 42). In step 43 targets are classified, giving each target a label such as “background” (for example parts of a car interior), “adult”, “infant”, “pet” etc. (item 44). This classification is done by examining both the spatial pattern and the temporal displacement data for each element within the target.

In step 45, the temporal displacement data of the elements within each human target are used to produce a spectral power distribution model, describing periodicities in the target's movement. In an embodiment of the invention, Welch's method is used to produce the spectral power density model (a non-parametric spectral model). In another embodiment, an (Auto Regressive Moving Average) ARMA model (a parametric spectral model) is used to produce the spectral power density model. Physiological parameters are estimated for human targets, including the breathing rate, heart rate and heart rate variability. Breathing rate and heart rate are estimated from the location of peaks in the spectral power distribution. In an embodiment, using Welch's method, heart rate variability is estimated from the width of the spectral peak corresponding to the heartrate. In another embodiment, using an ARMA model, the heart rate variability is estimated from the parametric representation of the ARMA model itself.

In step 47, the breathing rate, heart rate and heart rate variability are monitored for changes, indicating health or mental state changes.

In step 48, the 3D image associated with each element of a human target is used to identify the element with one or more human body parts. This identification is then used to generate additional data such as human posture and activity type (sitting, standing, running, etc.), as described hereinabove.

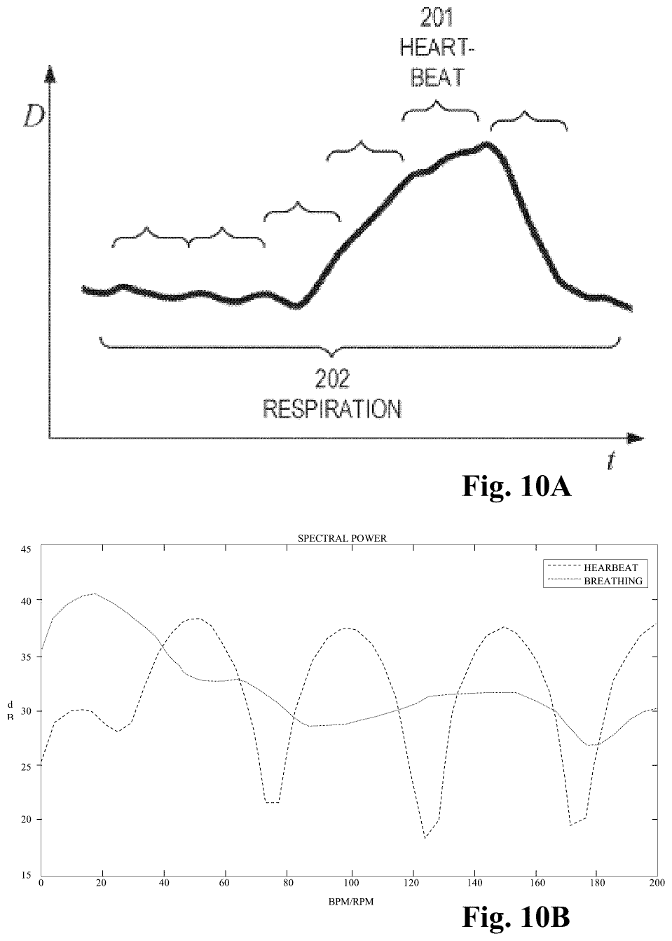

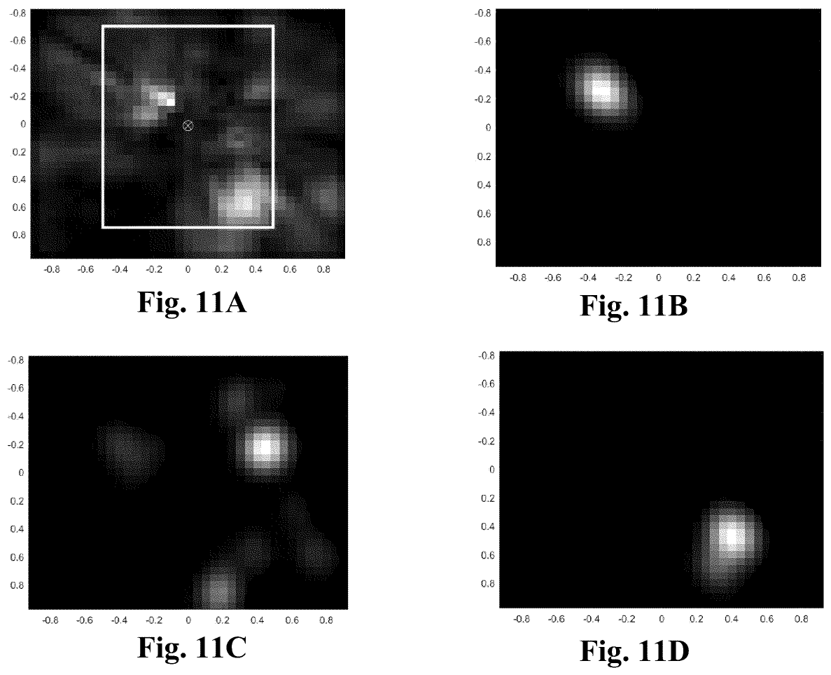

FIG. 10A shows a graph of radial displacement versus time, as measured for a human subject by a method and apparatus according to an embodiment of the present invention. A portion 201 shows a detected heartbeat, and a portion 202 shows a detected respiration. It is noted that according to this embodiment of the invention, it is not necessary to isolate the individual region of the body responsible for heartbeat and respiration.

FIG. 10B depicts a plot of the spectral power density of two elements identified by a method and apparatus according to an embodiment of the present invention.

In this embodiment, the sensor has been positioned close to a human subject, the two elements represent two modes of motion, one originating from the respiratory motion of the human subject, and the other originating from the heartbeat motion of the human subject. As can be seen, the elements represent motions which have different periodicity from one another. Each element is then used to calculate the corresponding rate parameter: breathing rate (measured in RPM—respirations per minute), and heart rate (measured in BPM—beats per minute).

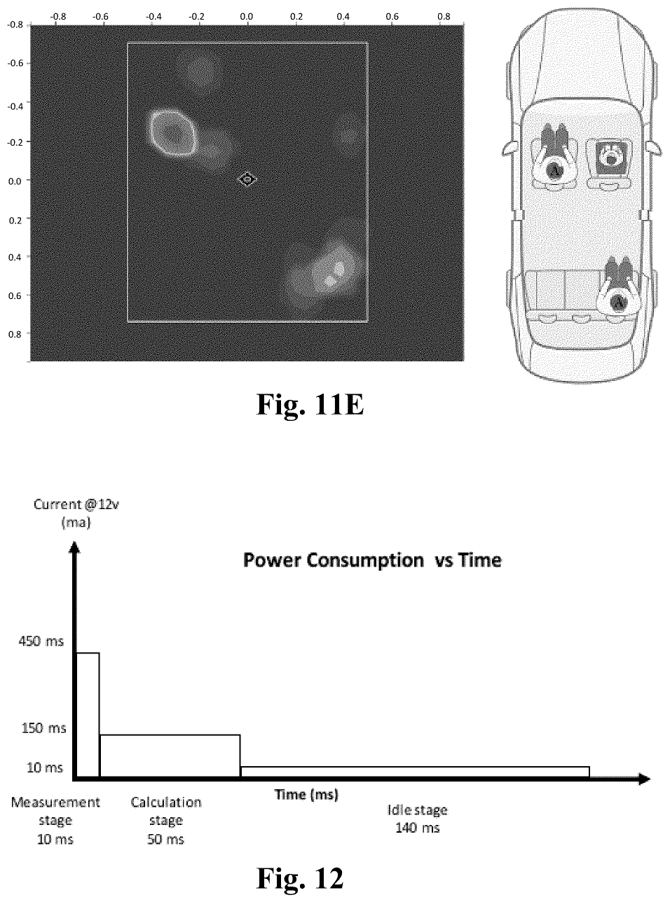

FIGS. 11A-E depict image products at various stages of processing of passengers sitting in a car environment.

By way of introduction, a car interior environment has several factors that contribute to the difficulty of identifying and separating passengers from one another and from the car interior background when imaging; passenger proximity, difference in passenger reflectivity, and car vibration.

Passenger proximity refers to passengers sitting next to each other and even contact each other, as is common in the back seat. Accordingly, these backseat passengers can appear as a single target object, when considering reflectance data of each frame separately.

The difference in passenger reflectivity can be very high due to difference in size (e.g. adult vs infant), positioning, and orientation. Differences in passenger reflectivity may degrade detection performance (false positive and false negative rate).

Car vibration also presents a significant challenge for current state of the art MIMO imaging techniques. The difficulty in detecting a change in position is exacerbated as passenger background (the car interior itself) vibrates and alters its reflective properties. As noted above, these imaging obstacles are resolved through the use of correlated motion as the differentiating parameter.

FIG. 11A depicts a 2D top view projection of a 3D image, generated by a MIMO radar installed in the roof of the passenger cabin of the car. The image represents a single captured frame. A white rectangle has been added to indicate the boundary of the car interior. The specific scenario being shown is that of an adult sitting in the driver seat (top left corner of the white rectangle), an infant sitting in the passenger front seat (top right corner of the white rectangle), and another adult sitting in the right seat in the back row (bottom right corner of the white rectangle). As can be seen, it is extremely difficult to identify the infant, due to its low reflectivity compared to the adult passengers. Objects associated with adult passengers mask the signal reflection from the infant.

FIG. 11B-D, show the spatial pattern associated with three elements which have been decomposed from a sequence of frames, by identifying the correlated motion of each individual passenger. These spatial patterns allow for an easy identification of three passengers.

FIG. 11E shows a screenshot of a user interface, used as an output of the system. On the left side is an image produced by filtering and then recombining the spatial patterns shown in FIGS. 8b, 8c, 8d. On the right side is a graphical summarization of the occupancy state reported by the system, correctly identifying the two adults and the infant in the correct positions. The classification of passengers into adults and infants is done by examining the spatial pattern for each detected element.

The separated components characterize the spatial movement modes associated with each type of movement, e.g. the spatial movement mode associated with respiration and the spatial movement mode associated with heartbeat.

The sets of voxels over which the movement is characterized can originate from a target tracking function, or it can originate form a priori knowledge, such as the candidate seating locations of persons in a car. The set of voxels may encompass multiple people, where the set of movement modes would encompass, for example, the respiration patterns of those multiple people. In the case of a moving vehicle, the spatial movement modes may include motion induced by the vibration of the vehicle, and the measured voxels may include reference objects such as empty seats. In other examples the measurement may include moving objects in the environment, such as ceiling fans, so as to separate fluctuations induced by such objects and movements induced by people of interest.

According to some embodiments, the system is be configurable to operate in various detection or activation modes; high detection mode, a medium modes, or a standby mode in which the fps and respective time durations are set by a user or manufacturer. Following are examples of activation modes:
High active mode: Capture rate of 30 frames per second (fps) for a period of 12 seconds, then a capture recess for 18 seconds, and repeating these two steps 6 sequential times (overall 3 minutes);
Medium active mode: capture rate of 10 fps for a period of 9 seconds, then a capture recess for 51 seconds, and repeating these two steps 10 sequential times (overall 10 minutes);
Standby mode: No capture for a period of 10 minutes, while former data captured and processed is saved for future analysis and comparison.

The system provides further flexibility by providing a configuration provision to activate in various activation modes, each for a predefined time or for a predetermined number of cycles or be activated by a combination of predefined time period and cycles.

Furthermore, according to some embodiments, the system can automatically change from one activation mode to another, responsively to collected data.

According to some embodiments, the system can be activated (turned “ON”) manually, and according to some embodiments, the system can be automatically activated responsive to predetermined instructions (for example during specific hours) and/or a predetermined condition of another system.

Additional power saving provisions include provisions to activate a reduced number of radar, transmit/receive modules, and processors in accordance with different power consumption or activation modes.

According to some embodiments, the system can be temporarily triggered OFF or temporarily activated in a “standby” mode, for power resources conservation.

FIG. 12 depicts operating stages of one operating cycle employed during active detection modes, in accordance with a certain embodiment. As shown, the operating cycle includes a measurement stage, calculation stage, and an idle stage.

In the measurement stage, capture of a complex target object employs a current of 450 mA via a voltage of 12v, for a time slot of 10 msec, at a full frame rate, for example between 15 to 25 frames per second (fps).

During the calculations stage, where calculations are executed in accordance with at least some of the above-mentioned methods to identify a motion, using a current of 150 mA via a voltage of 12v, for a time slot of 50 msec;

During the idle stage, a current of 30 mA via a voltage of 12v is employed, for a time slot of 140 msec to ensure memory retention previously captured or calculated data.

According to some embodiments, the methods and system mentioned above, can be implemented for various of monitoring and alerting uses. In a certain application, for example, a baby/toddler sitting in the back seat of a vehicle or in a crib is monitored. The device is configured to activate an alarm responsively to detection of a threshold variation change in breathing rate or heartbeat rate.

Another vehicular monitoring application is the field of detection of a baby or a toddler remaining in the vehicle after a threshold amount of time following engine disengagement and door locking.

In a certain embodiment the monitoring device is implemented within a vehicle to monitor occupants.

In a certain embodiment, the vehicular monitoring device is configured to be activated when the engine is OFF and/or the doors are locked in accordance with at least one of the above mentioned high and medium activation modes for a predetermined number of cycles and/or time period. The device is linked to the engine and locking system so at to provide such actuation functionality. Situations in which no motion is observed the monitoring device assumes a standby mode for a configurable time period.

According to some embodiments, the alert is selected from either activating the vehicle's horn, activating the vehicle's air ventilation, opening the vehicles windows, unlocking the vehicle, sending an alert to an application user, sending an alert to emergency services, and any combination thereof. According to some embodiments, alert is repeated until the system is manually switched OFF.

The monitoring device is configured to sequentially repeat the “monitoring” and “standby” operating modes, until: the vehicle is switched “ON”, wherein the system is either manually turned OFF, automatically turned off, or continues to monitor in accordance with either the passage of a threshold period of time or an achievement of a threshold number of repetitions.

Similarly, the device can be employed to the monitor the elderly or sick at one's bed and activate an alarm responsively to a threshold variation in breathing rate, heart rate, or heart rate variability.

The device-linked alarm includes audial alarms, visual alarms, or the combination of both, and in certain embodiments the alarm is activated remotely through any of a variety of wireless technologies.

It should be appreciated that embodiments formed from combinations of features set forth in separate embodiments are also within the scope of the present invention. Furthermore, while certain features of the invention have been illustrated and described herein, modifications, substitutions, and equivalents are included within the scope of the invention.

Aspects of the present disclosure relate to systems and methods for classifying vehicle occupants in the various seats of the vehicle.

Systems and methods intended for such tasks are required to be able to operate around the clock, including in the dark, and to detect and classify occupants even if concealed, such as covered by a blanket for example. Reliable classifications should be provided regardless of the car state, immaterial of whether the ignition is on, the air-conditioning is working, whether the vehicle is stationary or moving, and even if moving on a bumpy road.

Classification of occupants may include various categories such as, but not exclusively, age group, weight, size, indication whether a child seat exists, position of the occupant, animal vs. human, child vs. adult, male vs female as well as objects like water bottles and hanging shirts (which tend to move while driving) and the like.

Although classifying the occupants is important, it is also frequently required to simultaneously respect their privacy, and to avoid detecting and recording identifying details.

Possible technologies for classification occupants may include pressure sensors under the seats, a camera which may be assisted by a depth camera of any kind, a stand-alone depth camera, ultrasonic imaging and radar imaging.

The main drawback of cameras is the required external light source, and the inability to penetrate through non-transparent materials. Additionally, the image resolution of cameras may invade privacy.

Depth cameras which emit their own light in infrared frequencies for example, are capable of operating in darkness, but may be saturated during daytime. Additionally, they often do not penetrate through seats and blankets.

Pressure sensors provide information about weight, but do not provide any information about shape or size of the occupant, and therefore, they are generally insufficient.

On the other hand, radar and ultrasonic imaging systems can be implemented using waves with a wavelength in the order of 1 cm. Such systems are capable of operating in darkness and can penetrate objects which are not transparent to visible light. The wavelength of 1 cm is sufficient for classification of the occupants into groups such as, age group, weight, size, indication whether a child seat exists, position of the occupant, etc. . . . as outlined above to accord with technical and legal requirements and safety rules, but is insufficient to identify them. Radar and Ultrasonic sensors can be used to identify passengers under a blanket, and are not saturated by natural sources of light and sound.

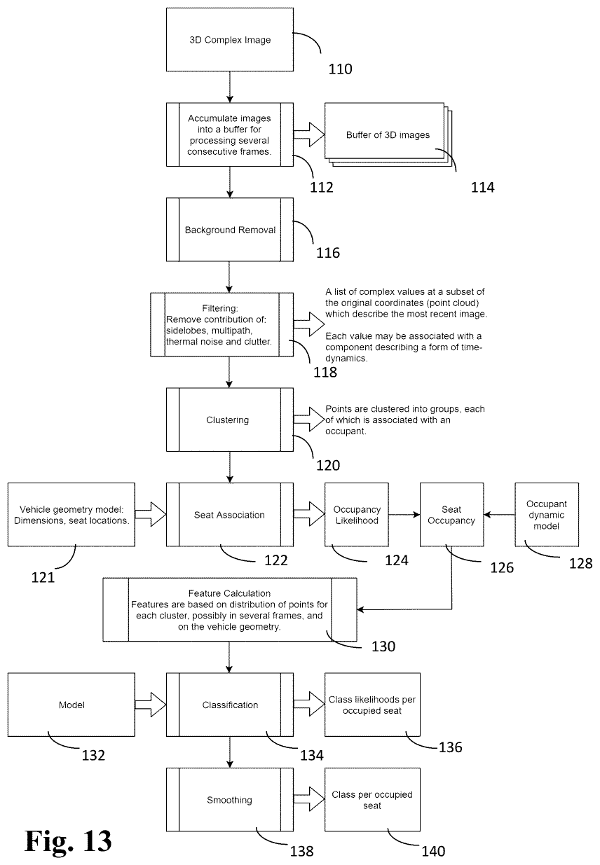

With reference to FIG. 13, a method is described for converting a 3D complex image obtained inside a vehicle over a grid of coordinates into a list of occupants of the vehicle with an associated class.

The method comprises obtaining a 3D complex image of the occupants of a vehicle cabin—step 110.

Image accumulation is required for obtaining a dynamic model of the occupants. The three dimensional image (complex values at a predefined set of coordinates) is stored as a row vector in a matrix. The matrix may store a predefined number of images (frames), or the number of frames to be stored may be variable.

An array of transmitting and receiving elements is used in order to generate a set of complex values associated with coordinates in a predefined area or volume inside and possibly around a vehicle. These values and associated coordinates are referred to as a complex 3D image. The magnitude of a complex value may indicate the probability that a reflecting object is located in that coordinate.

US Patent Publication 2019/0254544 titled DETECTING AND MEASURING CORRELATED MOVEMENT BY ULTRA-WIDEBAND MIMO RADAR incorporated herein by reference provides an exemplary method for obtaining a 3D complex-image of moving occupants is described. Another method is described in J. M. Lopez-Sanchez, J. Fortuny-Guasch, “3-D Radar Imaging Using Range Migration Techniques”, IEEE transactions on antennas and propagation, vol. 48, no. 5, May 2000, pp 728-737, which is incorporated by reference herein.

As a particular case, the image may store real values only, representing, for example the magnitude in each voxel.

A known algorithm for constructing such a complex image for an array of transmitting and receiving elements is the Delay and Sum algorithm (DAS). A variation on the DAS algorithm can be found in Giulia Matrone, Allesandro Stuart, Giosue Caliano, Giuvanni Magenes, “The Delay Multiply and Sum Algorithm in Ultrasound B-Mode Medical Imaging”, IEEE Transactions on Medical Imaging, Vol. 34, number 4, April 2015 which is incorporated herein by reference. More complex algorithms include algorithms for solving inverse problems. A review of solving inverse problems in imaging can be found in Alice Lucas, Michael Iliadis, Rafael Molina, Aggelos K. Katsaggelos, “Using Deep Neural Networks for Inverse Problems in Imaging: Beyond Analytical Methods”, IEEE Signal Processing Magazine, Volume 35, Issue 1, January 2018, pp. 20-36, as well as in Michael T. McCann, Kyong Hwan Jin, Michael Unser, “Convolutional Neural Networks for Inverse Problems in Imaging: A Review”, IEEE Signal Processing Magazine, Volume 34, Issue 6, Nov. 2017, pp. 85-95 both of which are also included by reference.

When classifying occupants, it may be assumed that they will show at least slight movement over time, such as chest movements and breathing, so phase variations over time for a given coordinate may indicate movement of the object. This can be detected when reviewing multiple frames. Images may be accumulated (step 112) in a buffer 114.

The walls of the cabin, seats and other constant and stationary features may be subtracted from the detected signals by a background removal algorithm—step 116.

Background removal may be achieved by as subtraction of the mean value for each coordinate, for example, in one or both of the following ways:
Applying a high-pass filter on each of the coordinates

For each column in the matrix of images, subtracting the mean value of the column.

Filtering—step 118 is performed to remove the contribution of sidelobes, multipath, thermal noise and clutter. The filtering step of FIG. 10 is performed based on dynamic behavior is expanded on in FIG. 11.

The points are then clustered into groups, each of which is associated with an occupant (step 120).

Data corresponding to the vehicle geometry model, dimensions and seat locations is provided—step 121. Each cluster is associated with a seat—step 122, and an occupation likelihood statistic is generated—step 124, such that a threshold value is used to decide whether or not a seat is occupied—step 126. This decision may be supplemented by results of an occupant dynamic model—step 128.

Features of each cluster are calculated, based on the vehicle geometry and the distribution of points for each cluster, possibly over several frames—step 130.

A model 132 is applied to the features classification of step 130 to create a classification 134 which assesses the likelihood that an occupied seat is assigned to a specific class, and this can be smoothed—step 138 to allocate the occupier of a seat to a specific class.

Occupation determination and classification procedure may involve various methods particularly machine learning. For example, a neural network may be trained to determine occupation or perform classification into required category groups, although other classification methods may be used. Parameters of the function may be optimized using a learning algorithm. Where the function is implemented in the form of a neural network, a feed forward neural network may be utilized where appropriate. Additionally or alternatively, a network with feedback may be used to take account of historical features such as an RNN (recurrent neural network), an LSTM (Long and Short Term Memory) network, or the like.

Alternatively, or additionally values for the coordinates of every box around a seat may be used as an input to a network, rather than a list of particular features. Accordingly, a convolutional neural network (CNN) may be appropriate. It will be appreciated that combinations of any of the above, such as a combined CNN with an RNN may be preferred. The values of coordinates within each box may be used for determining whether the seat associated with a particular box is occupied.

Although neural networks are described above for illustrative purposes, other classification algorithms may be additionally or alternatively used to provide the required classification, for example SVM (support vector machine), decision trees, an ensemble of decision trees, also known as decision forest. Other classification algorithms will occur to those skilled in the art.

### Decomposition (SVD)

Multipath, grating-lobes and side-lobes, have a similar dynamic behavior. Therefore singular value decomposition (SVD) tends to represent them using a single vector with a time varying coefficient.

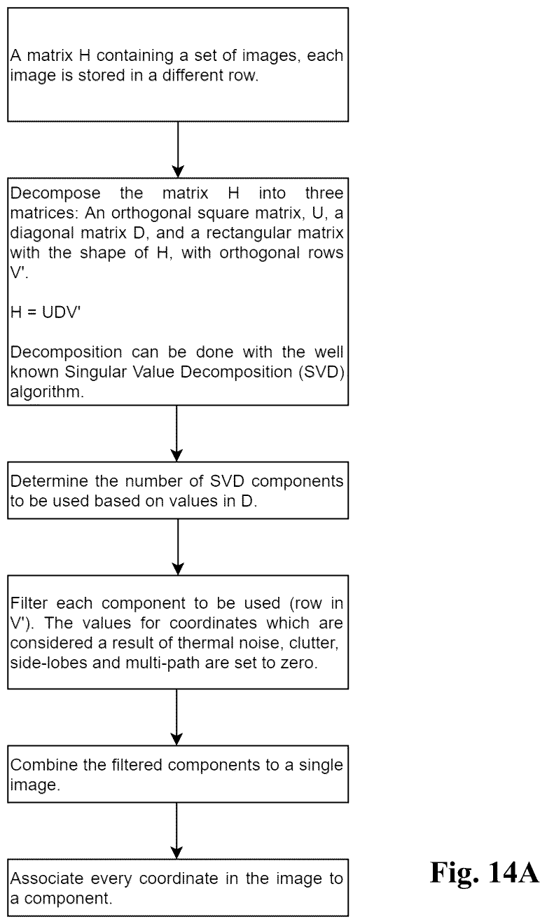

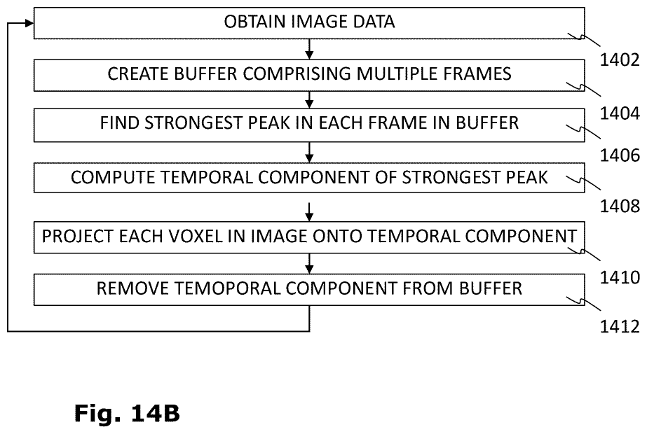

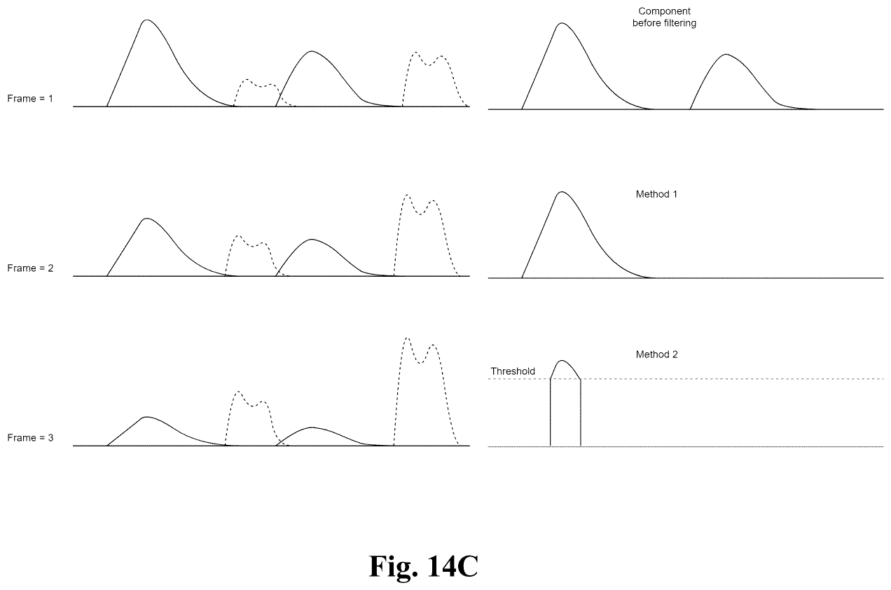

With reference to FIG. 14A, the time-evolution of a signal representing an image is shown. The set of images can be decomposed into two components, one of which is drawn with a solid line and the other with a dashed line. The magnitude of one component (solid line) decreases with time and the other increases with time. SVD decomposition can provide these components.

The mathematical formulation is as follows: Matrix H stores a set of images. Each row represents an image. the matrix H may be decomposed, for example, using a standard algorithm called singular value decomposition. The matrix is decomposed into three matrices.

H=U·D·V^{H}

In the decomposition, U represents a rotation matrix, D is a diagonal matrix (not necessarily square), and V^{H} is a matrix with equal dimensions to H with orthogonal rows. Rows of V^{H} contain components such as shown in the figure above.

### Determining the Number of Components

Determining the number of Required Components can be done with criteria based on distribution of the singular values, the values on the main diagonal of matrix D. One way is to select components which correspond to the largest singular values which add up to a percentage of the total value, for example 95%.

A different method is based on searching for a corner point in a graph of ordered singular values.

Both methods are known in the art.

### Alternative Decompositions

While SVD can be used for decomposition, this should be regarded as an example only for a broader class of decomposition options.

Alternative decompositions include, for example the following:
Independent Component Analysis (ICA)

This decomposition assumes that the observations are a linear mixture of statistically independent sources. The goal of the decomposition is to search for the independent sources.

It is formulated as follows:

$$( \begin{matrix}observations \\ {n}_{bservations} \times {n}_{dimensions}\end{matrix} ) + M \cdot ( \begin{matrix}sources \\ {n}_{sources} \times {n}_{dimensions}\end{matrix} )$$

Where M is an unknown mixing matrix. The ICA provides the sources which can be treated as components associated with occupants.

The inventors have noticed that at high SNR levels, performance of ICA often exceeds that of SVD in separating different occupants to different components or sources.

Still another example of an alternative decomposition method is Successive Spatio-Temporal Filtering Decomposition as illustrated in in the block diagram of FIG. 14B.

Image data is collected by the radar system as a series of frames 1402. The image frames may be stored in a memory unit of the processor as a buffer comprising a number of frames 1404, by way of example, a buffer may be a matrix including a set of 15 frames representing 3 seconds of data captured. The strongest peak in each frame may be determined 1406, for example the voxel with the strongest variance, or root-mean-squared value. Accordingly, the temporal component of the strongest peak may be determined for the strongest peaks in the buffer 1408 and each voxel of each frame may be projected onto the temporal component 1410. The temporal component may then be removed from the buffer 1412. The method may repeat as more image data is obtained.

### Component Filtering

Filtering each component is based on the assumption that a component, which describes a form of time-domain movement, should have localized energy. The filtering operation should preferably maintain localized values. In the following two filtering methods are described.

Method 1: Divide the image into high energy blobs and maintain only the blob with the highest energy.

Method 2: Maintain only coordinates with energy above a threshold. The threshold can be either relative to a peak value or absolute.

The two methods are depicted in FIG. 14C which demonstrates that the filtering operation zeros many of the coordinates. Generally, a mask can be defined, that decreases unwanted values instead of zeroing them.

### Combining Filtered Components to a Filtered Image

Combining the filtered components into an image can be done on a component by component basis.

The following notation is used herein for describing the method.
D=D where all elements except for (n,n) are set to 0.
\tilde{V}^{H}=Filtered components of V^{H}
\tilde{V}^{H}ₙ=n^{th} row of \tilde{V}^{H}
\tilde{H}ₖ⁽ⁿ⁾=k^{th} row of \tilde{H}⁽ⁿ⁾=U·\tilde{D}ₙ·\tilde{V}^{H}

One or more of several different methods may be used to generate a final image, as described below.

A first method involves averaging of the absolute value of several rows of \tilde{H}⁽ⁿ⁾:

$${image}^{( n )} = \frac{1}{C} \cdot {\sum }_{k ∈ rows of H} \vert {\tilde{H}}_{k}^{( n )} \vert$$

In the expression above, C represents a constant, for example, the number of rows in the sum operation. An alternative would be to average a power of the absolute values, for example the square of the absolute values. Another alternative is to give different weights to different rows of \tilde{H}⁽ⁿ⁾. Typically, rows are chosen which correspond to the most recently captured images.

An alternative method would be to use the component directly with no multiplication by U:

image⁽ⁿ⁾=|D(n,n)·\tilde{V}^{H} ₙ|

Multiplication by U provides the temporal information about the contribution of a component.

It is useful to associate each non-zero coordinate to a component. Association may be, for example, “hard” association, which means that it is associated with a single component, or “soft” association, which means assigning a probability that a coordinate is associated with a component.

Hard association may be formulated as follows: Coordinate i is associated with component n which maximizes the image at this coordinate.

$${component}_{i} = {arg min}_{n} \{ {image}_{i}^{( n )} \}$$

Soft association can be formulated as follows:

$$Pr \{ {component}_{i} = n \} = \frac{{image}_{i}^{( n )}}{{\sum }_{m} {image}_{i}^{( m )}}$$

Soft association tends to assist with clustering as performed in step 120. However, the association step is not mandatory. It can be skipped.

It will be appreciated that the position of the seats and the walls of the cabin are known. These fixed elements are background and may be removed. After removal of the background, the signals represent the occupants.

The purpose of clustering is to split the non-zero coordinates among occupants of the vehicle. This may be achieved by simply clustering the coordinates using standard clustering algorithms. An alternative approach is to make use of the a priori knowledge that different coordinates are associated with different components, where different coordinates are associated with different SVD (singular value decomposition) components by a hard association. For clarity, each component has a different color in the image.

Different occupants in a vehicle are expected to be separated into different SVD components, because they move differently in time. However, it is possible that a single SVD component will be associated with different occupants. In this case some of the coordinates within a component will form one cluster and others will form another cluster. Applying a clustering algorithm can be used to split this component into clusters.

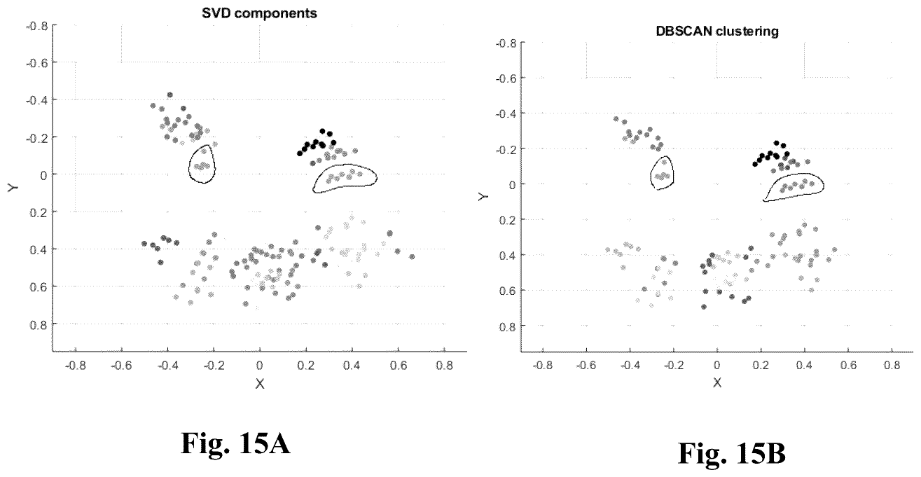

In FIG. 15A, two set of coordinates associated with the same SVD component were circled. Application of clustering per SVD component generates FIG. 15B, in which the circled clusters have been split to two components (different colors). FIG. 15B demonstrates per-component clustering using the DBSCAN algorithm, which also enables removing outliers. However, other clustering algorithms of the art may be used.

Referring back to FIG. 14C, two methods of filtering were described. Method 1 involved dividing the image into high energy blobs and maintaining only the blob with the highest energy. In contradistinction, method 2 maintained only coordinates with energy above a threshold which could be relative to a peak value or an absolute value.

Clustering using the DBSCAN algorithm is only necessary with the method 2, as method 1 leaves only one cluster per component.

Now, clustering may be applied to the clusters themselves. Each cluster is represented as a Gaussian distribution with mean and covariance which fit the distribution of points within the cluster. Distance metrics may then be defined between these Gaussian distributions.

Distance between distributions can take many forms. For example, Kullback Leibler divergence, Bhattacharyya distance, Hellinger distance and L2-norm of the difference

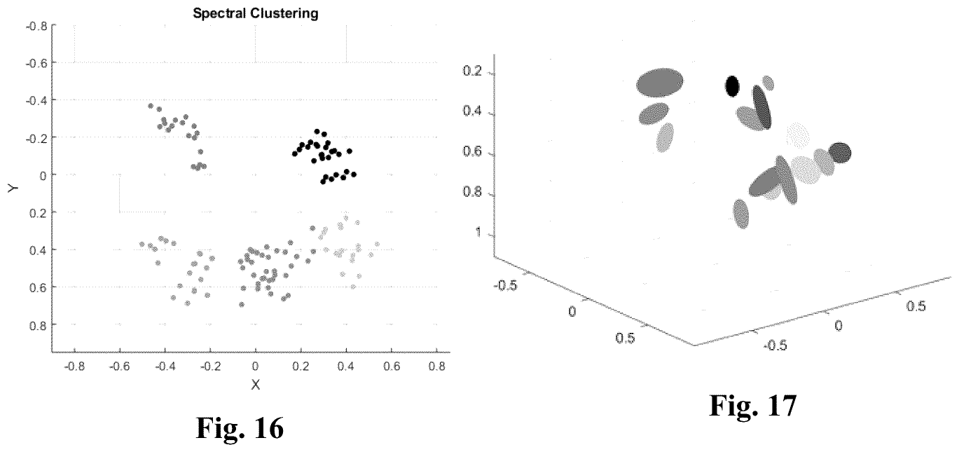

For example, spectral clustering which applies an algorithm known in the art, can be used to generate the results shown in FIG. 16, where five clusters are determined.

Fitting a Gaussian distribution to a collection of coordinates with given intensities can be done, for example, using the following equations. The notations are as follows.

The coordinates of a point in a cluster may be denoted by a column vector r=(rₓ, r_{y}, r_{z})^{T}. However, these coordinates are not necessarily Cartesian, and not necessarily three-dimensional. Every point in a cluster is associated with a magnitude, denoted by m. Magnitudes of cluster points are real and positive, that is mᵢ>0 for every point i.

A relative weight for each point in the cluster is defined, for example, by:

$${w}_{i} = \frac{{m}_{i}}{{\sum }_{i} {m}_{i}}$$

The center of the cluster is simply:

$$\underline{μ} = {\sum }_{i} {w}_{i} \cdot {\underline{r}}_{i}$$

A matrix is defined:

$$\tilde{c} = ( ( {c}_{1} ) ( {c}_{2} ) ( {c}_{p} ) )$$

where P denotes the number of points in a cluster, and

cᵢ=\sqrt{wᵢ}·(rᵢ−μ)

The covariance of the cluster is defined as

c=\tilde{c}·\tilde{c}^{T}

Finally, a Gaussian distribution can be defined using the covariance matrix c and the center μ:

$${f}_{Gauss} ( \underline{x} ) = \frac{1}{\sqrt{{( 2 π )}^{k} \cdot \vert c \vert}} \cdot {e}^{- \frac{1}{2} {( \underline{x} - \underline{μ} )}^{T} \cdot {c}^{- 1} \cdot ( \underline{x} - \underline{μ} )}$$

Other distributions may be used to describe a cluster of points, for example t-distribution, uniform distribution, Gaussian mixture, and so on.

FIG. 17 demonstrates representing clusters of points as Gaussians in a three-dimensional space.

It will be appreciated that each occupant of a vehicle may be associated with a seat. Seat association is the process of associating a seat in the vehicle to each occupant to determine whether a seat is occupied.

kₖ(r)=Pr{r∈seatₖ}

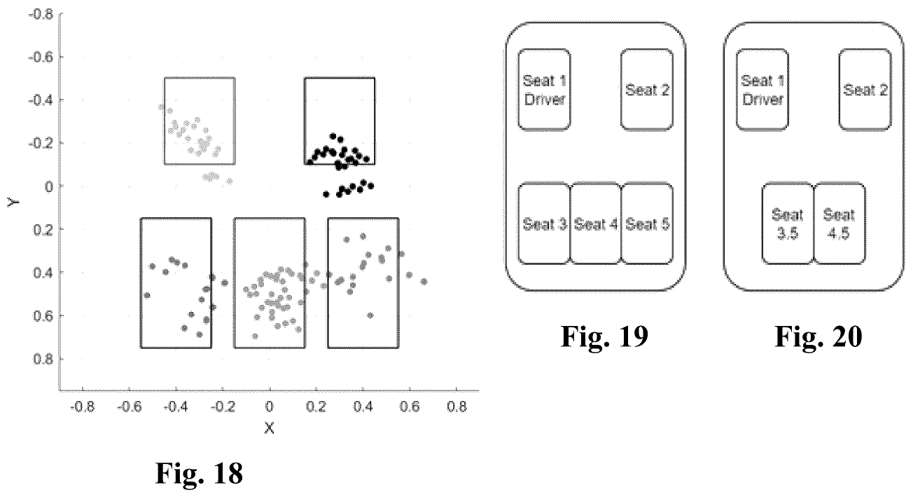

In FIG. 18, the rectangles define areas inside the vehicle associated with specific seats. Generally, the regions may be overlapping, and the distribution need not be uniform. However, as it is seen, the various clusters do align well with the boxes, and so it one can determine whether or not each seat is occupied.

$${t}_{k} ( {cluster}_{q} ) = {\prod }_{{\underline{r}}_{i} ∈ {cluster}_{q}} ( \frac{{p}_{k} ( {\underline{r}}_{i} )}{{\sum }_{{k}^{′}} {p}_{{k}^{′}} ( {\underline{r}}_{i} )} )$$

$${p}_{k} ( {cluster}_{q} ) = \frac{{t}_{k} ( {cluster}_{q} )}{{\sum }_{{k}^{′}} {t}_{{k}^{′}} ( {cluster}_{q} )}$$

In the second distance metrics may be used as described earlier in the chapter about clustering.

$${d}_{k} ( {cluster}_{q} ) = distance from distribution of cluster q to {p}_{k} ( \underline{r} ) .$$

$${p}_{k} ( {cluster}_{q} ) = \frac{{e}^{- {d}_{k}} ( {cluster}_{q} )}{{\sum }_{{k}^{′}} {e}^{- {d}_{{k}^{′}}} ( {cluster}_{q} )}$$

Once the probability that cluster q is associated with seat k are determined for every pair {k,q} each cluster (a) can be associated with the seat (k) for which pₖ(cluster_{q}) is maximal.

We distinguish between “hard” occupancy and “soft” occupancy. We shall now describe how occupancy is calculated.

For “hard” occupancy the following rules are used:
Seats with no clusters associated to them are considered empty seats.
Seats with at least one cluster associated to them are considered occupied.

For “soft” occupancy the following expression can be used for the probability that seat k is occupied as a function of the probability matrix p_{k′}(cluster₂) between each cluster q and each seat k′:

$$Pr \{ {Seat}_{k} is occupied \} =$$

$$1 - Pr \{ {Seat}_{k} is empty \} =$$

$$1 - {\prod }_{q} ( 1 - {p}_{k} ( {cluster}_{q} ) )$$

As an example of a model for valid transitions within a vehicle, consider a vehicle with 2 rows. In the front row in seat 1 is a driver, and beside him is seat 2. In the second row are seats 3, 4 and 5, where seat 3 is behind the driver. We may also define positions between two seats, such as seat 3.5 and seat 4.5.These arrangements are demonstrated in FIGS. 19 and 20.

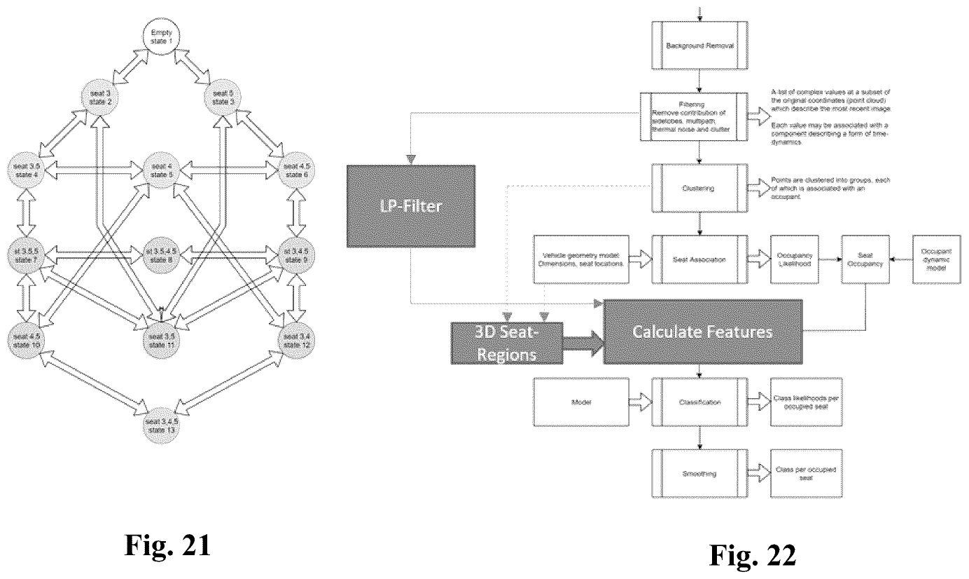

FIG. 21 demonstrates an occupancy transition diagram for the second row of FIG. 10. Each circle indicates an occupancy state, where the occupied seats and state number are indicated on the circle.

The occupancy probabilities Pr{Seatₖ is occupied} may be combined with the transition model in the following way.

A transition probability is assigned to each of the transitions in the diagram. For example for state 1, there is a probability of transition to state 2, a probability of transition to state 3, and a probability of remaining in state 1. These probabilities can be defined arbitrarily, or can be based on statistics. Wherever there is no connection between states in the diagram, it is assumed that the transition probability is 0. For example, the probability of transition from state 1 to state 5 is 0.

The transition probability from state s₁ to state s₂ is denoted by pₜ(s₁, s₂).

Each state s has occupied seats associated with it. The occupied seats associated with state s is denoted by oₛ. For example state 4 has seats 3 and 5 occupied: o₄={3,5}

Updating the probability of the system being in state s can be done as follows:

$${p}_{s} = {\prod }_{i ∈ {o}_{s}} Pr \{ seat i is occupied \} \cdot {\prod }_{i ∉ {o}_{s}} ( 1 - Pr \{ seat i is occupied \} ) \cdot {\sum }_{{s}^{′}} {p}_{{s}^{′}} {p}_{t} ( {s}^{′} , s )$$

In words, the probability of the occupancy being in state s is updated to the probability that the seats fit state s multiplied by the sum of transition probabilities to state s from states s′ multiplied by the probability of occupancy being in these states.

In some embodiments the exact probabilities are not calculated, but rather a number which is proportional to the probabilities.

In some embodiments the sum

$${\sum }_{{s}^{′}} {p}_{{s}^{′}} {p}_{t} ( {s}^{′} , s )$$

may be replaced by a single term which represents the most likelihood transition to state s as follows:

$${p}_{s} \approx {\prod }_{i ∈ {o}_{s}} Pr \{ seat i is occupied \} \cdot {\prod }_{i ∉ {o}_{s}} ( 1 - Pr \{ seat i i s occupied \} ) \cdot {max \{}_{{s}^{′}} {p}_{{s}^{′}} {p}_{t} ( {s}^{′} , s ) \}$$

In some embodiments the logarithm of the probabilities is used, and the multiplications above may be replaced by sums as follows:

$$log \{ {p}_{s} \} \approx {\sum }_{i ∈ {o}_{s}} log ( Pr \{ seat i is occupied \} ) + {\sum }_{i ∉ {o}_{s}} log ( 1 - Pr \{ seat i is occupied \} ) + log ( {max}_{{s}^{′}} \{ {p}_{{s}^{′}} , {p}_{t} ( {s}^{′} , s ) \} )$$

In some embodiments, the most likely state may be selected, and a few steps traced back according to the most likely consecutive states leading to it. Indicating a previous state that lead to the most likely current state stabilizes the system by reducing sensitivity to errors in the seat occupation probabilities.

Implementation of the last equation lends itself to linear programing methods.

With reference to FIG. 22, for the Occupant Classification, where required, a LP (low pass) filter may optionally be applied on the filtered 3D-Image for a configurable time constant (typically a few seconds). The LP filter may be a moving average or may be an IIR (infinite impulse response) exponential moving average filter. In other configurations, no low pass filter may be applied.

Use of an LP filter tends to maintain useful volumetric information (preserving voxels that account for reflections of the body). Different body parts may move at different times, and an LP filter may facilitate accumulating the information over time, giving a single 3D image.

The image is then divided into 3D regions, where each region encloses a single subject.

In the case of a vehicle, knowing its size and geometry, some measurements of the vehicle cabin can be utilized to derive the 3D regions.

For example for a 5 seat car, the following measurements are constant per car model relative to the sensor origin, and can be collected:
FRF—Front row seat foremost position (measured referring to seat “center of mass”—see image)
FRR—front row seat rearmost position (measured referring to seat “center of mass”—see image)
RRC—Rear row bench “center of mass”
BNCW—Rear row bench width
STH—seat height from ground to edge
SSH—Sensor height from ground to sensor

Another option is to use the decision of an upper layer that derives the number of occupants and the location (providing [x,y] coordinates for each occupant, and opening a box around that location [x—dx, x+dx, y—dy, y+dy, 0, SSH]).

Yet another option is to use the SVD decomposition to “color” each voxel by component number giving an initial guess for the clustering of voxels. Then each component can be put through DBSCAN—Density Based Spatial Classification of Applications with Noise (geometric) clustering to remove outliers and separate geometrically independent clusters to separate components. From this step on, each component (cluster) can no longer be separated and additional clustering is done to separate into cluster groups

Per 3D region, volumetric and intensity based features may be extracted.

Hand crafted cluster features may be utilized to assess the identity of an occupant.

### Notation

Given a 3D image region, all voxels that have an intensity above a certain level are extracted. This gives a list of the occupied voxels of the 3D Region.

voxelᵢ=[xᵢ, yᵢ, zᵢ, Iᵢ], i=1 . . . N

PC={voxel₁ . . . voxel_{N}}

From this list of points the following features are calculated. The coordinates are relative to a defined center-point of the 3D-Region. In the case of a car-seat, the center-point is defined to be directly on the seat (z-coordinate) and in the region where an adult's center of intensity is expected to be (x-, y-coordinate).

### Number of Occupied Voxels

The number of occupied voxels may indicate the volume of the region that is occupied.

Number of occupied voxels=N

### Center of Intensity

The center of intensity is the average position of the voxels weighted by their intensity

$$C = [ {C}_{x} , {C}_{y} , {C}_{z} ] = \frac{{\sum }_{i} ( {I}_{i} \cdot [ {x}_{i} , {y}_{i} , {z}_{i} ] )}{{\sum }_{i} {I}_{i}}$$

### Covariance and Weighted Covariance

The covariance gives a measure of how the points are distributed in space. It the case of occupant-classification it indicates the posture of the person (e.g. leaning forward adult/baby in an infant-seat)

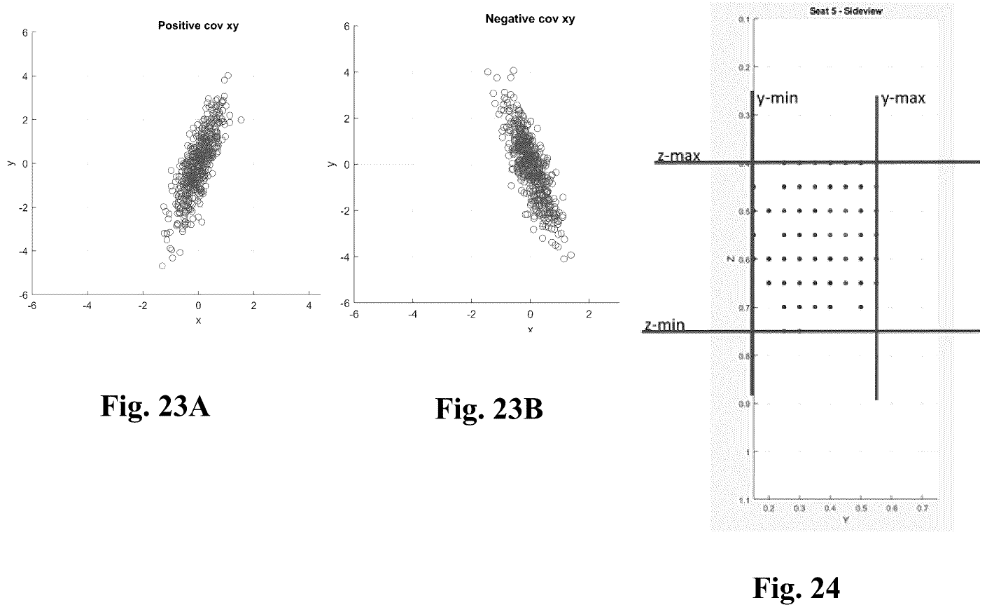

FIGS. 23A and 23B illustrate this principle.

The following algorithm shows an example of code for calculating weighted covariance.

numDimensions=3;

gmm_params.mean=zeros(numDimensions, 1);

gmm_params.cov=zeros(numDimensions, numDimensions, 1);

weights=iSeat_pts_Intensity/sum(iSeat_pts_Intensity);

gmm_params.mean=sum(weights.*iSeat_pts_XYZ)';

zero_mean_weighted_points=sqrt(weights).*(iSeat_pts_XYZ-gmm_params.mean');

gmm_params.cov=(zero_mean_weighted_points'*zero_mean_weighted_points);

$${weight}_{i} = \frac{{I}_{i}}{{\sum }_{i = 0}^{N} {I}_{i}}$$

$${zmwp}_{i} = \sqrt{{weight}_{i}} \cdot ( [ {x}_{i} , {y}_{i} , {z}_{i} ] - C )$$

$$co v = {zmwp}^{T} \cdot zmwp$$

### Extrema of Coordinates in 3D-Region

The extreme (maximum/minimum) coordinates of all 3 Cartesian axes give a measure of the region that is occupied in the 3D-Region.

With reference to FIG. 24, by drawing a rectangle around a cluster one can obtain an indication of size and shape of the occupant.

Xₘᵢₙ=min(X), X=x₁ . . . x_{N}

Xₘₐₓ=max(X), X=x₁ . . . x_{N}

Yₘᵢₙ=min(Y), Y=y₁ . . . y_{N}

Yₘₐₓ=max(Y), Y=y₁ . . . y_{N}

Zₘᵢₙ=min(Z), Z=z₁ . . . z_{N}

Zₘₐₓ=max(Z), Z=z₁ . . . z_{N}

### Center of Intensity for Defined z-Value

For an adult, the center of intensity on z-slices over the seat height is typically close to the backrest of the seat, for an infant in a rearward-facing baby seat the center of mass in certain slices is typically shifted more to the front.

$${weight}_{i} = \frac{{I}_{i}}{{\sum }_{i = 0}^{N} {I}_{i}} ∀ i ∈ \{ 1 … N \} s . t . {z}_{i} = e . g . 0.6 m over seat$$

$${zmwp}_{i} = \sqrt{{weight}_{i}} \cdot ( [ {x}_{i} , {y}_{i} , {z}_{i} ] - C ) ∀ i ∈ \{ 1 … N \} s . t . {z}_{i} = e . g . 0.6 m over seat$$

$$co v = {zmwp}^{T} \cdot zmwp$$

### Mean Intensity

$$\overline{I} = \frac{1}{N} {\sum }_{i = I}^{N} {I}_{i}$$

### Max Intensity

Iₘₐₓ=max(I), I=I₁ . . . I_{N}

### Energy Below the Seat Level

The energy below the seat level can indicate if an infant is on the seat. In case of an infant being on this seat there are no reflections from below the sitting height expected.

I_{belowseat}=sum(Iᵢ)∀i∈{1 . . . N}, zᵢ>seatheight

To stabilize the classification output, the classification may be saved to a buffer of a few seconds and a majority vote or a stabilizer with hysteresis may be used to determine the final classification-decision.

As noted hereinabove externally orientated vehicle mounted radar sensors may be used to provide object sensing in the surroundings of the vehicle to which the radar sensors are mounted. Various sensors may be used to sense objects around vehicles. Indeed, with the increased usage of autonomous vehicles such as self-driving cars and the like a plethora of sensors are used to detect objects in the vicinity of travelling vehicles. For example, sensors such as video cameras, ultrasonic sensors, infrared, LIDAR sensors and the like may be used to provide information pertaining to the environment through which the vehicle is travelling.

The application of radar is becoming more and more popular with the development of the RFIC and signal technology. Radar sensors have the advantage of operating in total darkness, fog, mist and rain. Radar is an electronic system with the advantages of low cost, low-power consumption, and high precision. The working efficiency of a radar system is based upon reliable and stable radar signal with wide coverage, high directionality, high gain and low signal-to-noise ratio.

The usefulness of radars as vehicle mounted sensors depends upon various factors, such as their resolution and accuracy of determining direction, range and speed as well as the identification and characterization of objects.

Various devices have been suggested for enhancing signal reflections in the radar range. For example, U.S. patent application Ser. No. 13/950,719 titled “Wearable radar reflectors” describes a wearable radar reflector which uses a retroreflector configured to reflect radiation received from a vehicle. Similarly U.S. patent application Ser. No. 17/294, 161 titled “Saddle riding type vehicle comprising a collision risk reduction system” describes a system for reducing a collision risk comprising at least one active radar reflector, which allows the radar visibility of the vehicle to be increased as a radar target by increasing its equivalent radar cross section. Although systems such as the abovementioned suggest methods for increasing the visibility of reflected signals, the signals reflected are not themselves enhanced and provide no new information regarding the object from which they are reflected.

It is particularly noted that beam enhancement systems and methods such as described herein may be used for enhancing radar reflections so as to provide useful information to vehicle mounted receiving radar.

Reference is now made to FIG. 25A which schematically represents an example of a vehicle mounted radar unit 600 configured to sense objects in the region 620 surrounding a vehicle to which it is mounted. The radar unit 600 may be mounted to various vehicles as required such as road vehicles such cars, trucks, bikes, trailers, caravans and the like, work vehicles such as diggers, cranes, and the like as well aircraft and watercraft where appropriate.

By way of example, the radar unit 600 may be mounted to a car and used to detect various objects in its vicinity such as other cars 621, bicycles 622, pedestrians 623, road signs 624, walls 625, curbs 626, trees 627 and the like.

Accordingly, the radar unit 600 may be used to harvest information regarding the environment through which the vehicle is travelling. This disclosure teaches various techniques that a radar unit may analyze data received such that useful information may be gathered such as the vehicles relative speed and the identification of hazards in its surroundings.

Referring now to the block diagram of FIG. 25B selected elements are presented of a possible radar system for sensing the surroundings of a vehicle. The system includes a radar unit 610 including a radar transmission unit 612 and a radar receiving unit 614.

The radar transmission unit 612 includes an array of transmitter antennas TX connected to an oscillator 616 and configured to transmit electromagnetic waves into a region surrounding the vehicle. The radar receiving unit 614 includes at least one receiver antenna RX connected to a processor 630 and configured to receive electromagnetic waves reflected by objects within the region surrounding the vehicle 620 and may be operable to generate raw data.

The processor 630 may include various modules such as a modulated reflection detector configured to identify and decode enhanced reflections received by the reflection unit 614.

Further processor units such as self-velocity calculation modules, wall detection modules, dynamic range enhancement modules, double reflection detection modules and the like may be included as described in the applicant's co-pending U.S. patent application Ser. No. 18/135,784 and which is incorporated herein by reference in its entirety.

A processor unit may be in communication with the radar receiving unit 614 so as to receive raw data from the radar unit 614 and generate environmental information based upon the received data.

The radar typically includes at least one array of radio frequency transmitter antennas and at least one array of radio frequency receiver antennas. The radio frequency transmitter antennas are connected to an oscillator (radio frequency signal source) and are configured and operable to transmit electromagnetic waves towards the target region. The radio frequency receiver antennas are configured to receive electromagnetic waves reflected back from objects within the target region.

Accordingly the transmitter may be configured to produce a beam of electromagnetic radiation, such as microwave radiation or the like, directed towards a monitored region such as an enclosed room or the like. The receiver may include at least one receiving antenna or array of receiver antennas configured and operable to receive electromagnetic waves reflected by objects within the monitored region.

The raw data generated by the receivers is typically a set of magnitude and phase measurements corresponding to the waves scattered back from the objects in front of the array. Spatial reconstruction processing is applied to the measurements to reconstruct the amplitude (scattering strength) at the three dimensional coordinates of interest within the target region. Thus each three dimensional section of the volume within the target region may represented by a voxel defined by four values corresponding to an x-coordinate, a y-coordinate, a z-coordinate, and an amplitude value.

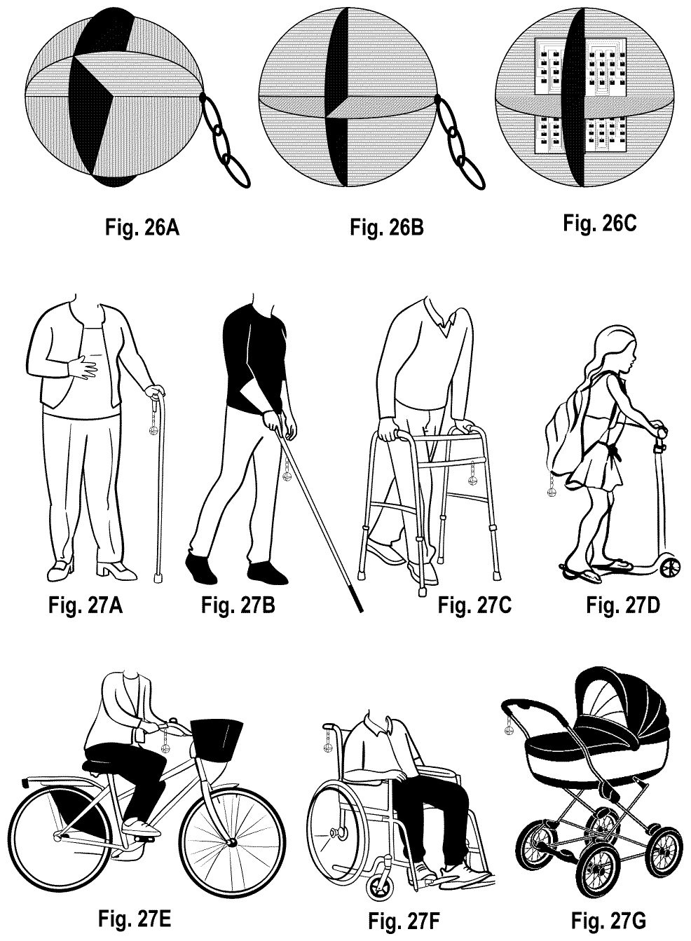

Referring now to FIGS. 26A and 26B, which illustrate two views of a possible retroreflector consisting of three co-orthogonal circular radar wave reflectors arranges such that they form eight corner cube reflectors configured to reflect all incident radar waves back in the at the same angle from which they are incident towards direction from which they came. The retroreflectors may further include an attachment means by which it may be attached to object to an intensify the reflections therefrom.

Referring now to FIG. 26C, which illustrates a further embodiment of a wavelength specific retroreflector incorporating wavelength specific retroreflectors such as described above in relation to FIG. 26C. Accordingly, the wavelength specific retroreflectors may be selected such that the bandwidth of enhanced reflection may serve as coded information, for example it may be indicative of the identity of the object to which the retroreflector is attached.

It will be appreciated that such reflectors may be connected to various bodies such as shown in FIGS. 27A-G so as to render them more visible to radar detectors. It is further noted that, where required, the placement of such reflectors may be selected specifically to provide a characteristic reflection having a spatial distribution indicative of the identity of the objection to which it is attached.

FIGS. 27A-G illustrate various examples of enhanced reflectors attached variously to walking stick, a white cane, a walking frame, a school bag, a bicycle, a wheelchair, a baby buggy or the like. It is a feature of the current invention that each of the enhanced reflectors may be selected to uniquely identify the body to which it is attached so as to communicate that information to the radar.

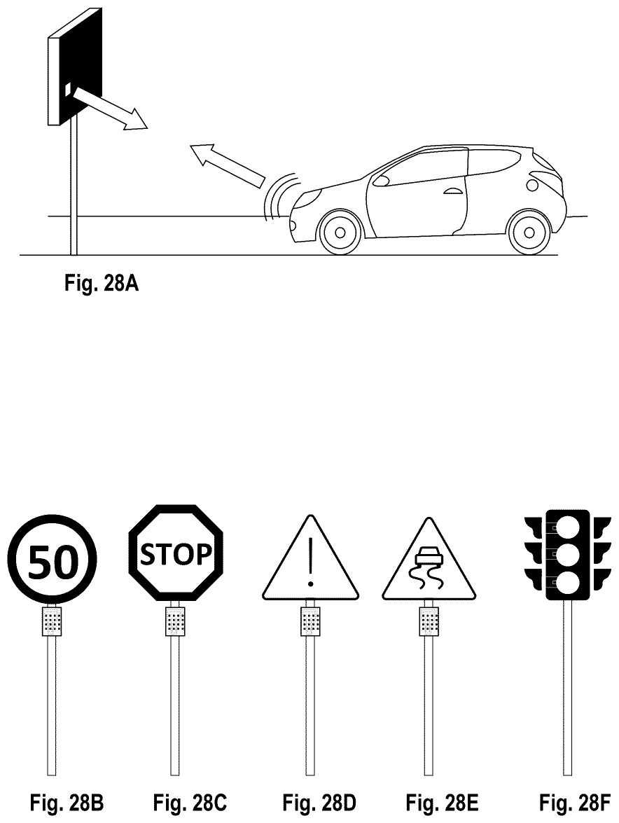

Reference is now made to FIG. 28A which schematically illustrates a radar enabled vehicle approaching an enhanced reflecting road sign according to an embodiment of the current invention. The radar enable vehicle includes a mounted radar which transmits radiation into its surroundings and receives signals reflected back from roadside objects. When radiation is incident upon the enhanced reflecting road sign the enhanced reflection retroreflectors may used to modulate the signal to carry characteristic information relating to the identity of the road sign.

A radar may transmit radiation at multiple frequencies by sweeping through a frequency range. The radar detector may be configured and operable to detect the intensity of the reflected signal. Accordingly, when an enhanced reflected signal is detected, the frequency of the enhanced reflection may indicate the particular wavelength specific retroreflector producing the reflection. By carefully selecting the bandwidth of the wavelength specific retroreflector connected to a given body, coded information may be communicated to the radar perhaps pertaining to the identity of the body to which it is connected or the shielded region into which it deflects the radio beam.

FIGS. 28A-E represent a selection of enhanced road signs each including a retroreflector uniquely identifying the road sign, for example as a 50 mph speed limit, a stop sign, a hazard warning, a slippery road surface warning or the like.

FIG. 28F represents a traffic light which may include multiple retroreflectors configured to represent three distinct states of the traffic light, RED-state, YELLOW-state and GREEN-state.

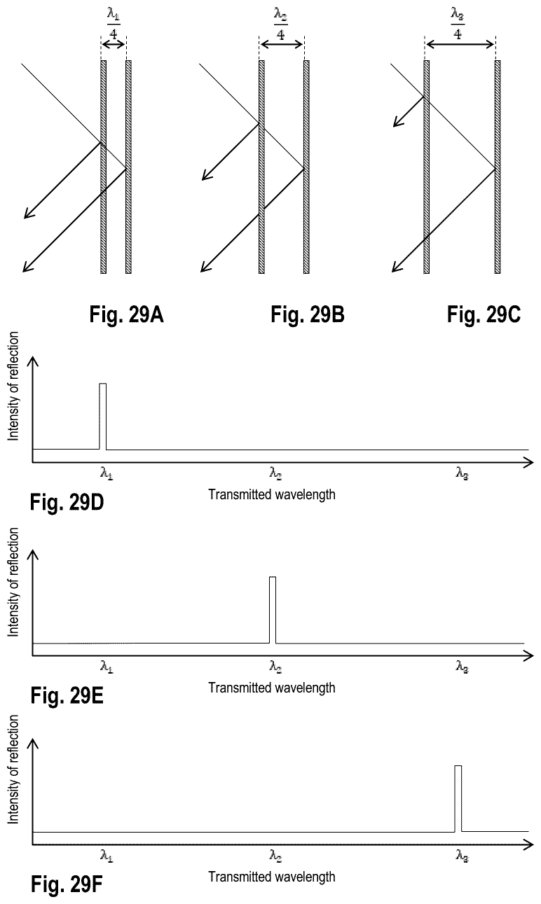

By way of example FIGS. 29A-C represent examples of wavelength specific retroreflectors which may be used to indicate three states of a traffic light for example. FIGS. 29D-F illustrate the expected reflection profiles from each of the wavelength specific retroreflectors of FIGS. 29A-C.

The first wavelength specific retroreflector of FIG. 29A comprises two parallel planar radar reflectors spaced at a first spacing distance of λ1/4. Accordingly, incident radiation with a first wavelength of λ1 is reflected back strongly. The corresponding response profile for the retroreflector of FIG. 29A is illustrated in FIG. 29D. A transmitted signal sweeping through multiple transmission frequencies and the intensity of the reflected signal displays a clear peak intensity reflection at the first wavelength λ1 which is indicative of the first wavelength specific retroreflector.

Similarly, the second wavelength specific retroreflector of FIG. 29B having a second plate spacing distance of λ2/4, has a corresponding response profile displaying a clear peak intensity reflection at the second wavelength λ2. Likewise, the third wavelength specific retroreflector of FIG. 29C having a third plate spacing distance of λ3/4, has a corresponding response profile displaying a clear peak intensity reflection at the third wavelength λ3.

The three characteristic frequencies may be indicative of the three states of a traffic light for example. Where appropriate, all three reflectors may be mounted to the traffic light and operable to orientate themselves as required to match the corresponding state of the light indications.

Alternatively, an active variable wavelength specific retroreflector such as illustrated in FIG. 5B may be used to vary the reflector plate spacing as required. The variable wavelength specific retroreflector has a variable plate spacing λ(t)/4 allowing the enhanced wavelength λ(t) to be adjusted over time to encode various signals as required.

### Technical Notes

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

The scope of the disclosed subject matter is defined by the appended claims and includes both combinations and sub combinations of the various features described hereinabove as well as variations and modifications thereof, which would occur to persons skilled in the art upon reading the foregoing description.

What is claimed is:

1. A vehicle cabin monitoring system comprising:
a radar unit comprising:
at least one transmitter antenna connected to an oscillator and configured to transmit electromagnetic waves into the vehicle cabin, and
at least one receiver antenna configured to receive electromagnetic waves reflected by objects within the vehicle cabin and operable to generate raw data;
at least one beam deflection device configured to direct the electromagnetic waves transmitted by the at least one transmitter antenna towards at least one obstructed region within the vehicle cabin and further configured to direct the electromagnetic waves reflected by objects within the obstructed region towards the at least on receiver antenna.

2. The vehicle cabin monitoring system of claim 1 wherein the at least one beam deflection device comprises an outbound radio beam generator comprising a radio wave deflector operable to redirect incoming radio waves into an outbound radio beam.

3. The vehicle cabin monitoring system of claim 1 wherein the at least one beam deflection device is selected from at least one of a group consisting of: a reflector, a refractor, a dielectric lens, a grating, a reflectarray and combinations thereof.

4. The vehicle cabin monitoring system of claim 1 wherein the at least one beam deflection device has a time-varying reflectivity.

5. The vehicle cabin monitoring system of claim 1 wherein the at least one beam deflection device comprises a switched impedance.

6. The vehicle cabin monitoring system of claim 1 wherein the at least one beam deflection device comprises a controlled dielectric material.

7. The vehicle cabin monitoring system of claim 1 wherein the at least one beam deflection device comprises a movable element.

8. The vehicle cabin monitoring system of claim 1 wherein the at least one beam deflection device is incorporated into a ceiling section of the vehicle cabin selected from a group consisting of a sunroof, a headliner, a roofing beam, a vehicle pillar, a roll bar and combinations thereof.

9. The vehicle cabin monitoring system of claim 1 wherein the at least one beam deflection device is incorporated into at least one of: a windshield of the vehicle, a rearview mirror of the vehicle, and a pillar of the vehicle.

10. The vehicle cabin monitoring system of claim 1 wherein the at least one beam deflection device is incorporated into a head restraint.

11. The vehicle cabin monitoring system of claim 1 wherein the at least one beam deflection device further comprises a modulator operable to modulate incoming radio wave signals.

12. The vehicle cabin monitoring system of claim 1 wherein the modulator comprises a temporal modulator operable to adjust reflectivity over time.

13. The vehicle cabin monitoring system of claim 1 wherein the modulator comprises a temporal modulator operable to adjust gain over time according to a gain function.

14. The vehicle cabin monitoring system of claim 1 wherein the modulator comprises a temporal modulator operable to adjust beam-phase over time according to a phase adjustment function.

15. The vehicle cabin monitoring system of claim 1 wherein further comprises a reflectivity modulation unit configured to adjust the reflectivity of the outbound radio beam generator so as to encode information therein.

16. A method for monitoring a vehicle cabin:
providing a radar unit comprising:
at least one transmitter antenna connected to an oscillator and configured to transmit electromagnetic waves into the vehicle cabin, and
at least one receiver antenna configured to receive electromagnetic waves reflected by objects within the vehicle cabin and operable to generate raw data;
providing at least one beam deflection device within the vehicle cabin;
the at least one transmitter antenna transmitting electromagnetic waves into the vehicle cabin;
the at least one beam deflection device directing the electromagnetic waves transmitted by the at least one transmitter antenna towards at least one obstructed region within the vehicle cabin;
objects within the at least one obstructed region within the vehicle cabin reflecting the electromagnetic waves;
receiving electromagnetic waves reflected by objects within the vehicle cabin; and
the at least one beam deflection device directing electromagnetic waves reflected by objects within the obstructed region towards the at least on receiver antenna.

17. The method for monitoring a vehicle cabin of claim 16 further comprising:
providing a processor unit configured to receive raw data from the radar unit and operable to generate image data based upon the received data;
providing a memory unit configured and operable to store the image data; and
providing at least one output unit; and
generating a set of complex values associated with voxels within the vehicle cabin including the obstructed region.

18. The method for monitoring a vehicle cabin of claim 17 further comprising:
converting the set of complex values into a 3D complex image;
filtering the 3D images by removing contribution of at least one of sidelobes, multipath, thermal noise and clutter;
detecting occupancy of seats within the vehicle cabin.

19. The method for monitoring a vehicle cabin of claim 18 further comprising:
categorizing at least one occupant of a seat within the vehicle cabin;
detecting a posture of at least one occupant of a seat within the vehicle cabin; and
determining a seatbelt status for at least one occupant of a seat within the vehicle cabin.

20. The method for monitoring a vehicle cabin of claim 18 further comprising the output unit cancelling air bag operation if unsafe.
